(19) United States
(12) Patent Application Publication
(10) Pub. No.: US 2008/0193414 A1
(43) Pub. Date: Aug. 14, 2008

(54) Use of an Immunoglobulin Domain-Containing Cell Surface Recognition Molecule For Treating Diseases

(76) Inventors: Amanda Proudfoot, Chens Sur Leman (FR); Bruno Antonsson, Billiat (FR); Linda Kontula, Geneva (CH); Francis Vilbois, Minzier (FR)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/913,620

(22) PCT Filed: May 4, 2006
(86) PCT No.: PCT/EP06/62063

§ 371 Date: Nov. 5, 2007

Related U.S. Application Data
(60) Provisional application No. 60/681,651, filed on May 17, 2005.

(30) Foreign Application Priority Data
May 6, 2005 (EP) 05103791.9

(51) Int. Cl. A61K 38/17; A61K 38/21; A61K 31/7088; A61K 39/44; A61K 48/00; A61P 31/12; A61P 31/00; A61P 33/00; A61P 37/00; A61P 35/00; A61P 9/00; A61P 7/00

(52) U.S. Cl. 424/85.6; 514/12; 514/13; 514/8; 514/11; 514/44; 424/178.1; 424/85.4; 424/93.2

(57) ABSTRACT

The invention relates to the use of INSP052 for treatment and/or prevention of infectious disease, properdin-related disease, MBL2-related disease, MASP1-related disease, MASP2-related disease, Antithrombin III-related disease, Complement factor H-related disease and/or Albumin-related disease. Combinations of INSP052 with an interferon, a TNF antagonist or a further anti-infectious or anti-blood clotting agent are also within the present invention.

### FIELD OF THE INVENTION

The invention relates to the use of INSP052 for the treatment and/or prevention of infectious disease, properdin-related disease, MBL2-related disease, MASP1-related disease, MASP2-related disease, Antithrombin III-related disease, Complement factor H-related disease and/or Albumin-related disease.

### BACKGROUND OF THE INVENTION

The protein INSP052 was disclosed in WO2003/093316 and International Application No. PCT/GB2004/004772 as an immunoglobulin domain-containing cell surface recognition molecule, and more particularly, as a cytokine antagonist.

### SUMMARY OF THE INVENTION

The invention is based on the unexpected finding that INSP052 interacts with proteins of the complement pathway, namely properdin, mannose-binding lectin C (MBL-C), MASP1, MASP2, antithrombin III, complement factor H and albumin.

It is therefore a first object of the invention to use INSP052 for the preparation of a medicament for the treatment and/or prevention of infectious disease, properdin-related disease, MBL2-related disease, MASP1-related disease, MASP2-related disease, Antithrombin III-related disease, Complement factor H-related disease and/or Albumin-related disease. It is a second object of the invention to use a cell expressing INSP052, or an expression vector comprising the coding sequence of INSP052, for the preparation of a medicament for the treatment and/or prevention of infectious disease, properdin-related disease, MBL2-related disease, MASP1-related disease, MASP2-related disease, Antithrombin III-related disease, Complement factor H-related disease and/or Albumin-related disease. The present invention is also directed towards the use of INSP052 for the preparation of a pharmaceutical composition for the treatment and/or prevention of infectious disease, properdin-related disease, MBL2-related disease, MASP1-related disease, MASP2-related disease, Antithrombin III-related disease, Complement factor H-related disease and/or Albumin-related disease.

### DESCRIPTION OF THE INVENTION

The invention is based on the unexpected finding that INSP052 interacts with proteins of the complement pathway, namely properdin, mannose-binding lectin C (MBL-C), MASP1, MASP2, antithrombin III, complement factor H and albumin. These surprising properties presently characterized of the polynucleotides or the corresponding polypeptides of WO2003/093316 and International Application No. PCT/GB2004/004772 make them particularly suitable for the preparation of a medicament or of a pharmaceutical composition.

In a first aspect, the invention therefore relates to the use of an INSP052 polypeptide for the preparation of a medicament for the treatment and/or prevention of infectious disease, properdin-related disease, MBL2-related disease, MASP1-related disease, MASP2-related disease, Antithrombin III-related disease, Complement factor H-related disease and/or Albumin-related disease, wherein said polypeptide is selected from the group consisting of:
a) A polypeptide consisting of SEQ ID NO: 16, or
b) A polypeptide comprising any of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 27, SEQ ID NO: 28, SEQ ID NO: 29, SEQ ID NO: 30, SEQ ID NO: 31, SEQ ID NO: 32 or SEQ ID NO: 33, or
c) A soluble form consisting of any of SEQ ID NO: 20, SEQ ID NO: 22, SEQ ID NO:27, SEQ ID NO: 28, SEQ ID NO: 29, SEQ ID NO: 30, SEQ ID NO: 31, SEQ ID NO: 32 or SEQ ID NO: 33, or
d) A mature form consisting of any of SEQ ID NO: 22, SEQ ID NO: 24 or SEQ ID NO: 26, or
e) A histidine tag form consisting of SEQ ID NO: 29, or
f) A glycosylated form of any of the polypeptides of (a) to (e), wherein the polypeptide is glycosylated at one or more sites, or
g) A mutein of any of the polypeptides of (a) to (f), wherein the amino acid sequence has at least 40% or 50% or 60% or 70% or 80% or 90% identity to at least one of the corresponding sequences in (a) to (f), and retaining INSP052 biological activity, or
h) A mutein of any of the polypeptides of (a) to (f) wherein any changes in the amino acid sequence are conservative amino acid substitutions to the amino acid sequences in (a) to (f), and retaining INSP052 biological activity, or
i) A salt or an isoform, fusion protein, functional derivative, active fraction or circularly permutated derivative of any of the polypeptides of (a) to (h).

In a second aspect, the invention relates to the use of an INSP052 nucleic acid molecule for the preparation of a medicament for the treatment and/or prevention of infectious disease, properdin-related disease, MBL2-related disease, MASP1-related disease, MASP2-related disease, Antithrombin III-related disease, Complement factor H-related disease and/or Albumin-related disease, wherein said nucleic acid is selected from the group consisting of:
a) A nucleic acid sequence as set forth in any of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, or SEQ ID NO: 25, or
b) A nucleic acid sequence which hybridizes to the complement of the nucleic acid sequence of (a) under moderately stringent conditions or under highly stringent conditions, or
c) A nucleic acid sequence of any of (a) or (b) wherein said nucleic acid sequence encodes an amino acid sequence having conservative amino acid substitutions to the amino acid sequences in any of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 27, SEQ ID NO: 28, SEQ ID NO: 29, SEQ ID NO: 30, SEQ ID NO: 31, SEQ ID NO: 32 or SEQ ID NO: 33.

In a third aspect, the invention relates to the use of an INSP052 polypeptide for the preparation of a pharmaceutical composition for the treatment and/or prevention of infectious disease, properdin-related disease, MBL2-related disease, MASP1-related disease, MASP2-related disease, Antithrombin III-related disease, Complement factor H-related disease and/or Albumin-related disease, wherein said polypeptide is selected from the group consisting of:
a) A polypeptide consisting of SEQ ID NO: 16, or
b) A polypeptide comprising any of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 27, SEQ ID NO: 28, SEQ ID NO: 29, SEQ ID NO: 30, SEQ ID NO: 31, SEQ ID NO: 32 or SEQ ID NO: 33, or
c) A soluble form consisting of any of SEQ ID NO: 20, SEQ ID NO: 22, SEQ ID NO:27, SEQ ID NO: 28, SEQ ID NO: 29, SEQ ID NO: 30, SEQ ID NO: 31, SEQ ID NO: 32 or SEQ ID NO: 33, or
d) A mature form consisting of any of SEQ ID NO: 22, SEQ ID NO: 24 or SEQ ID NO: 26, or
e) A histidine tag form consisting of SEQ ID NO: 29, or
f) A glycosylated form of any of the polypeptides of (a) to (e), wherein the polypeptide is glycosylated at one or more sites, or
g) A mutein of any of the polypeptides of (a) to (f), wherein the amino acid sequence has at least 40% or 50% or 60% or 70% or 80% or 90% identity to at least one of the corresponding sequences in (a) to (f), and retaining INSP052 biological activity, or
h) A mutein of any of the polypeptides of (a) to (f) wherein any changes in the amino acid sequence are conservative amino acid substitutions to the amino acid sequences in (a) to (f), and retaining INSP052 biological activity, or
i) A salt or an isoform, fusion protein, functional derivative, active fraction or circularly permutated derivative of any of the polypeptides of (a) to (h).

In a fourth aspect, the invention relates to an INSP052 nucleic acid for the preparation of a pharmaceutical composition for the treatment and/or prevention of infectious disease, properdin-related disease, MBL2-related disease, MASP1-related disease, MASP2-related disease, Antithrombin III-related disease, Complement factor H-related disease and/or Albumin-related disease, wherein said nucleic acid is selected from the group consisting of:
a) A nucleic acid sequence as set forth in any of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, or SEQ ID NO: 25, or
b) A nucleic acid sequence which hybridizes to the complement of the nucleic acid sequence of (a) under moderately stringent conditions or under highly stringent conditions, or
c) A nucleic acid sequence of any of (a) or (b) wherein said nucleic acid sequence encodes an amino acid sequence having conservative amino acid substitutions to the amino acid sequences in any of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 27, SEQ ID NO: 28, SEQ ID NO: 29, SEQ ID NO: 30, SEQ ID NO: 31, SEQ ID NO: 32 or SEQ ID NO: 33.

Preferably, a soluble INSP052 is used for the preparation of a medicament or of a pharmaceutical composition.

The term “soluble INSP052” or “sINSP052” herein refers to an INSP052 polypeptide which is not membrane bound or to an INSP052 polypeptide which doesn't contain one or more transmembrane domains.

It will be appreciated by the person skilled in the art that in accordance with the present invention, a substance which stimulates release or potentiates the activity of endogenous INSP052 can equally be used for treatment and/or prevention of infectious disease, properdin-related disease, MBL2-related disease, MASP1-related disease, MASP2-related disease, Antithrombin III-related disease, Complement factor H-related disease and/or Albumin-related disease.

The invention is based on the unexpected finding that a soluble INSP052 consisting of the extracellular part of the membrane-bound INSP052 (SEQ ID NO: 22) interacts with proteins of the complement pathway, namely properdin, mannose-binding lectin C (MBL-C), MASP1, MASP2, antithrombin III, complement factor H and albumin.

The term “complement-pathway protein” herein refers to as protein selected from properdin, mannose-binding lectin C (MBL-C), MASP1, MASP2, antithrombin III, complement factor H and albumin. These complement-pathway proteins are associated with infectious disease, properdin-related disease, MBL2-related disease, MASP1-related disease, MASP2-related disease, Antithrombin III-related disease, Complement factor H-related disease and/or Albumin-related disease.

Preferably, the infectious disease is selected from Systemic Fungal Disease, Rickettsial Disease, Chlamydial Disease, Parasitic Infection, Viral Disease, Abscess, Human Immunodeficiency Virus Infection, Bacteremia, Septic Shock, Sexually Transmitted Disease or Bacterial Disease.

Preferably, the bacterial disease is selected from a disease caused by Gram-Positive Cocci, caused by Gram-Negative Aerobic Cocci, caused by Gram-Positive Bacilli, caused by Gram-Negative Bacilli, caused by Anaerobic Bacilli, caused by Spirochetes or caused by Mycobacteria.

In one embodiment, a “Gram-Negative Aerobic Cocci” refers herein to an organism of the genus Neisseria including N. meningitidis, N. gonorrhoeae, and numerous saprophytic Neisseria sp that commonly inhabit the oropharynx, vagina, or colon.

Preferably, the disease caused by Gram-Negative Aerobic Cocci is selected from meningitis, bacteremia, urethritis, cervicitis, proctitis, pharyngitis, salpingitis, epididymitis, gonorrheal infection, acute cacterial meningitis or Meningococcal infection.

“Bacteremia” herein refers to bacteria in the bloodstream.

“Septic shock” herein refers to sepsis with hypoperfusion and hypotension refractory to fluid therapy. “Sepsis” or “systemic inflammatory response syndrome” herein refers to a serious infection, localized or bacteremic, that is accompanied by systemic manifestations of inflammation. Sepsis due to bacteremia herein refers to septicemia.

Preferably, the Parasitic Infections is selected from Extraintestinal Protozoa infection, infection with Free-Living Amebas, Intestinal Protozoa infection, Nematode (Roundworm) Infection, Trematode (Fluke) infection or Cestodes (Tapeworms) infection.

Preferably, the Extraintestinal Protozoa infection is malaria.

Preferably, the Viral disease is selected from Respiratory Viral Disease, Herpesvirus Infection, Central Nervous System Viral Disease, Arbovirus or Arenavirus Disease.

In addition, each complement-pathway protein has been shown to be associated with specific diseases, as described below.

As such, in one embodiment, the invention related to the use of INSP052 for the preparation of a medicament or of a pharmaceutical composition for the treatment of a properdin-related disease.

In one embodiment, the invention relates to the use of INSP052 for the preparation of a medicament or of a pharmaceutical composition for the treatment of a MBL2-related disease.

In one embodiment, the invention relates to the use of INSP052 for the preparation of a medicament or of a pharmaceutical composition for the treatment of a MASP1-related disease.

In one embodiment, the invention relates to the use of INSP052 for the preparation of a medicament or of a pharmaceutical composition for the treatment of a MASP2-related disease.

In one embodiment, the invention relates to the use of INSP052 for the preparation of a medicament or of a pharmaceutical composition for the treatment of an Antithrombin III-related disease.

In one embodiment, the invention relates to the use of INSP052 for the preparation of a medicament or of a pharmaceutical composition for the treatment of a Complement factor H-related disease.

In one embodiment, the invention relates to the use of INSP052 for the preparation of a medicament or of a pharmaceutical composition for the treatment of an Albumin-related disease.

Properdin (factor P; 469 amino acids; 51276 Da) is a plasma protein that is active in the alternative complement pathway of the innate immune system. It is a positive regulatory factor that binds to many microbial surfaces to stabilize the C3b,Bb convertase. The C3b, Bb convertase then rapidly cleaves more C3 to C3b, which acts either as an opsonin or to reinitiate the pathway in an amplification loop that proceeds on the bacterial cell, but not on the host cell. In the alternative pathway, C3 is thus activated through factor B, factor D, and properdin P, under the control of factors I and H. Deficiency of properdin is associated in particular with a heightened susceptibility to Neisseria species. Defects in properdin are the cause of properdin deficiency (pfd), resulting in higher susceptibility to bacterial infections; especially to meningococcal infections. Three phenotypes have been reported: complete deficiency (type I), incomplete deficiency (type II), and dysfunction of properdin (type III). Properdin has been linked to many diseases as set forth by the term “properdin-related disease”.

A “properdin-related disease” is selected from complement deficiency, complete deficiency (type I), incomplete deficiency (type II), dysfunction of properdin (type III), meningococcal infection, Neisseria infection, hypocomplementemia, afibrinogenemia, acute poststreptococcal glomerulonephritis, agammaglobulinemia, agammaglobulinemia swiss type, glomerular disease, ametropia, wiskott-aldrich syndrome, IgA glomerulonephritis, protein c deficiency, nephritis, chronic granulomatous disease, bacterial infection, proteinuria, septic shock, hemolysis, systemic infection, septicemia, immunologic deficiency syndrome, systemic lupus erythematosus, malaria, thrombosis, diabetes mellitus, necrosis, acquired immunodeficiency syndrome.

Preferably, the glomerular disease is selected from nephritic syndrome, nephrotic syndrome, primary glomerular disease, or secondary renal disease.

Preferably, the primary glomerular disease is selected from minimal change disease, focal segmental glomerulosclerosis, membranous glomerulonelhritis, membranoproliferative glomerulonephritis, mesangial proliferative glomerulonephritis, IgA nephropathy, rapidly progressive glomerulonephritis, or fibrillary glomerulonephritis.

Preferably, the nephritic syndrome is selected from hematuria, hypertension, renal insufficiency, edema, acute glomerulonephritis, transient glomerulonephritis, postinfectious glomerulonephritis, fulminant glomerulonephritis, rapidly progressive glomerulonephritis (RPGN), indolent Glomerulonephritis, IgA nephropathy, Crescentic glomerulonephritis, Pauci-immune RPGN, Immune complex RPGN, Anti-GBM antibody disease autoimmunity, primary renal hematuric-proteinuric syndrome, asymptomatic hematuric-proteinuric syndrome, chronic nephritic-proteinuric syndrome, chronic glomerulonephritis, slowly progressive glomerular disease.

Mannose-binding lectin 2 gene (MBL2; 248 amino acids; 26143 Da; Subunit: oligomeric complex of 6 set of homotrimers) encodes a mannose-binding lectin (MBL), or protein (MBP), that is secreted by the liver as part of the acute-phase response and is involved in innate immune defense. The ligands for MBL are expressed by a wide variety of microorganisms, and binding of the protein leads to opsonization resulting in susceptibility to frequent and chronic infections as well as activation of the complement system. MBL2 has been linked to many diseases as set forth by the term “MBL2-related disease”.

A “MBL2-related disease” is selected from vascular disease, atherosclerotic disease, mannose-binding protein deficiency, chronic infection due to opsonin defect, meningococcal infection, systemic lupus erythematosus, complement deficiency, acute-phase reaction, meningococcal meningitis, IgA glomerulonephritis, IgG subclass deficiency, cystic fibrosis, cutaneous subacute lupus erythematosus, rheumatoid arthritis, immunologic deficiency syndrome, respiratory tract infection, swollen joint, influenza, primary sjogrens syndrome, hemolysis, cystic fibrosis, bacterial infection, mucocutaneous lymph node syndrome, common variable immunodeficiency, lupus nephritis, polyarthritis, inflammation, septic shock, tuberculosis, virus infection, pneumococcal infection, hiv infection, otitis media, nephritis, systemic infection, malaria, membranous glomerulonephritis, sepsis syndrome, adenocarcinoma of the colon, hepatitis c, spontaneous abortion, sjogrens syndrome, acute otitis media, glomerulonephritis, herpes simplex, dermatomyositis, necrosis, aspergillosis, atherosclerosis, epstein-barr virus infection, pneumonia, primary biliary cirrhosis, hepatitis b, liver disease, osteomyelitis, cardiovascular disease, hepatitis, neutropenia, sarcoidosis, acquired immunodeficiency syndrome, cirrhosis, meningitis, colitis ulcerative, glioma, kidney disease, coronary arteriosclerosis, chronic kidney failure, colorectal cancer, shock, Kawasaki disease, Acute respiratory tract infection, vulvar vestibulitis syndrome, Behcet's disease, Crohn's disease, Restenosis, human t-cell leukemia virus type i (hTLV-i) provirus infection, dermatomyositis, HBV infection, IgA nephropathy, COPD infections, gestational Diabetes Mellitus, giant cell arteritis, dental caries, Chlamydia trachomatis infection, chorioamnionitis, Primary Biliary Cirrhosis, Chlamydia pneumoniae infection, celiac disease, post-Q fever fatigue syndrome, or chronic Q fever.

Preferably, the cardiovascular disorder is selected from Cardiac and Respiratory Arrest, Valvular Heart Disease, Arterial Hypertension, Endocarditis, Orthostatic Hypotension, Syncope, Pericardial Disease, Arteriosclerosis, Cardiac Tumor, Coronary Artery Disease, Disease of the Aorta and Its Branches, Heart Failure, Peripheral Vascular Disorder, Shock, Athletic Heart Syndrome or Arrhythmia.

The Ra-reactive factor (RARF) is a complement-dependent bactericidal factor that binds to the Ra and R2 polysaccharides expressed by certain enterobacteria. RARF activity is found in the sera of a diverse group of vertebrates, suggesting that it is an evolutionarily conserved mechanism to resist infection by these bacterial strains. RARF includes a 100-kD component, CRARF, also called MASP1 or p100, that was thought to activate the complement components C4 (C4F; C4S), C2, and C3. Subsequent work, however, separated MASP1 from MASP2 and showed that MASP1 activates C3 and C2, whereas MASP2 activates C4 and C2. The other component of RARF is mannan-binding lectin, a plasma protein member of the complement system that binds to microbial carbohydrates and activates the MASPs. The MASPs then recruit C4 and C2 to generate the C3 convertase or directly activate C3.

Alternate splicing of MASP1 results in two transcript variants encoding two RARF components that are involved in the mannan-binding lectin (MBL) pathway of complement activation. Each isoform is cleaved into two chains which form a heterodimer linked by a disulfide bond. The encoded proteins are members of the trypsin family of peptidases. MASP1 (699 amino acids; 79258 Da) is therefore a component of the bactericidal Ra-reactive factor RARF which specifically binds to Ra and R2 polysaccharides expressed by certain enterobacteria. MASP1 has been linked to a few diseases as set forth by the term “MASP1-related disease”.

A “MASP1-related disease” is selected from glomerulonephritis, IgA glomerulonephritis, systemic lupus erythematosus or immunologic deficiency syndrome.

MASP2 (686 amino acids; 75685 Da; Isoform 2 binds to MASP-1; belongs to the peptidase S1 family) is a trypsin protease that presumably plays an important role in the initiation of the mannose-binding lectin (MBL) complement activation pathway. After activation it cleaves C4 generating C4A and C4B. MASP2 has been linked to diseases as set forth by the term “MASP2-related disease”.

A “MASP2-related disease” is selected from immunologic deficiency syndrome or MASP2 deficiency.

Preferably, an “immunologic deficiency sydrome” is selected from acquired immunodeficiency syndrome or immunodeficiency linked to the Mannose-binding protein (MBP) locus.

Antithrombin III (464 amino acids; 52602 Da; belongs to the serpin family) is the most important serine protease inhibitor in plasma that regulates the blood coagulation cascade. AT-III inhibits thrombin as well as factors IXa, Xa and XIa. Its inhibitory activity is greatly enhanced in the presence of heparin. Defects in antithrombin III are the cause of antithrombin iii deficiency (at-iii deficiency). AT-III deficiency is a form of thrombophilia, an autosomal dominant disorder in which affected individuals are prone to develop serious spontaneous thrombosis. AT-III deficiency is classified into 4 types. Type I is characterized by a 50% decrease in antigenic and functional levels. Type II has defects affecting the thrombin-binding domain. Type III is an alteration of the heparin-binding domain. Plasma at-iii antigen levels are normal in type II and III. Type IV consists of miscellaneous group of unclassifiable mutations. AT-III Basel, Tours/Alger/Amiens/Toyama, Rouen-1, -2, -3 and -4, have decreased (or lack) heparin-binding properties. At-iii Hamilton, Glasgow/Sheffield/Chicago, Northwick-Park/Milano-1, Pescara, Denver/Milano-2, and Utah are deprived of inhibitory activity.

Antithrombin III has been linked to diseases as set forth by the term “Antithrombin III-related disease”.

An “Antithrombin III-related disease” is selected from a hematology-related disorder, antithrombin III deficiency, thrombophilia, protein s deficiency, activated protein c resistance, disseminated intravascular coagulation, thrombosis, protein c deficiency, venous thrombosis, familial antithrombin III deficiency, blood coagulation disorder, thromboembolism, congenital dysfibrinogenemia, deep vein thrombosis of lower limb, arterial thrombosis, hemorrhage, hypercortisonism, mesenteric vein thrombosis, pre-eclampsia, cerebral thrombosis, purpura fulminans, pulmonary embolism, nephrotic syndrome, systemic infection, fibrinolytic defect, antiphospholipid syndrome, cerebral venous thrombosis, factor XII deficiency, heIIp syndrome, portal vein thrombosis, pulmonary thromboembolism, multiple organ failure, superior sagittal sinus thrombosis, venous occlusion, postoperative hemorrhage, homocystinuria, septic shock, abruptio placentae hyperhomocysteinemia, thrombocytopenia, spontaneous platelet aggregation disease, renal vein thrombosis, veno-occlusive disease, cerebral infarction, pregnancy toxemia, postphlebitic syndrome, thrombocytosis, livedo reticularis, atherosclerosis, platelet dysfunction, hepatic veno-occlusive disease, stasis, retinal vein occlusion, promyelocytic acute leukemia, shwartzman phenomenon, skin necrosis, liver cirrhosis, increased bleeding time disorder, sneddon syndrome, polycythemia vera, purpura, eclampsia, hemophilia a, sepsis syndrome, legg-perthes disease, myocardial infarction, cerebrovascular accident, meningococcemia, inherited blood coagulation disorder, angina pectoris, mediastinal fibrosis, thrombophlebitis, cardiovascular disease, fetal growth retardation, pulmonary vein trombosis, hyperlipoproteinemia, vascular disease, liver dysfunction, hepatic vein thrombosis, acute myocardial infarction, hemorrhagic fever with renal syndrome, infarction, central retinal vein occlusion, liver disease, hematoma subcutaneous, Behcets syndrome, acute lymphocytic leukemia, hypoprothrombinemia, mesenteric infarction, proteinuria, cerebral embolism, myeloproliferative disorder, habitual abortion, disseminated intravascular coagulation sepsis, thromboembolism, Marburg virus infection, Ebola virus infection or Thrombosis Burns.

Preferably, the hematology-related disorder is selected from Anemia, Histiocytic Syndrome, Iron Overload related disorder, Leukemia, Lymphoma, Myeloproliferative Disorder, Plasma Cell Dyscrasia, Hemostasis and Coagulation Disorder, Disorder of the Spleen, Thrombotic Disorder, Platelet Disorder, Vascular Bleeding Disorder, Leukopenia, Lymphocytopenia or AIDS-Associated Hematologic Disorder and Malignancy.

There is an interest to develop drugs related to Antithrombin III. For example, Advantek Biologics has tried to develop a drug with an antithrombin III peptide for genetic disorder. Aventis Behring has discontinued to work on an antithrombin III peptide for disseminated intravascular coagulation sepsis. Bayer has launched an antithrombin III drug for thromboembolism. GTC Biotherapeutics has pre-registered a transgenic antithrombin III for Marburg virus infection, Ebola virus infection and Thrombosis Burns. Myriad Genetics has tried to develop MPC-1203 (Coagulation inhibitor Antithrombin III) for thrombosis.

Complement factor H (1231 amino acids; 139125 Da) is a member of the Regulator of Complement Activation (RCA) gene cluster and encodes a protein with twenty short concensus repeat (SCR) domains. Alternate transcriptional splice variants, encoding different isoforms, have been characterized. This protein is secreted into the bloodstream and has an essential role in the regulation of complement activation, restricting this innate defense mechanism to microbial infections. Factor H functions as a cofactor in the inactivation of C3b by factor I and also increases the rate of dissociation of the C3bBb complex (C3 convertase) and the (C3b)NBB complex (C5 convertase) in the alternative complement pathway. Mutations in this gene have been associated with hemolytic-uremic syndrome (HUS) and chronic hypocomplementemic nephropathy. HUS is a microvasculature disorder leading to microangiopathic hemolytic anemia associated with distorted erythrocytes (‘burr cells’), thrombocytopenia, and acute renal failure. Both dominant and recessive modes of inheritance have been reported. Most cases of HUS are associated with epidemics of diarrhea caused by verocytotoxin-producing bacteria, but atypical cases of HUS not associated with diarrhea (aHUS) also occur. Complement Factor H is associated with Membroproliferative glomerulonephritis and Factor H deficiency. Complement factor H has been linked to diseases as set forth by the term “Complement factor H-related disease”.

A “Complement factor H-related disease” is selected from uremic syndrome, thrombotic thrombocytopenic purpura, hemolytic-uremic syndrome, hemolytic microangiopathic anemia, membranoproliferative glomerulonephritis, hypocomplementemia, primary hyperoxaluria, acute kidney failure, thrombocytopenia, cancer of bladder, hemolytic anemia, antiphospholipid syndrome, hemolysis, systemic lupus erythematosus, inflammation, malignant neoplasm, liver disease, thrombosis, malignant neoplasm of lung, chronic hypocomplementemic nephropathy, Factor H deficiency or Age-Related Macular Degeneration.

Albumin (ALB; 609 amino acids; 69366 Da) is a soluble, monomeric protein which comprises about one-half of the blood serum protein. Albumin functions primarily as a carrier protein for steroids, fatty acids, and thyroid hormones and plays a role in stabilizing extracellular fluid volume (regulation of the colloidal osmotic pressure of blood). Serum albumin has a good binding capacity for water, Ca(2+), Na(+), K(+), fatty acids, hormones, bilirubin and drugs. Mutations in this gene on chromosome 4 result in various anomalous proteins. Albumin is synthesized in the liver as preproalbumin which has an N-terminal peptide that is removed before the nascent protein is released from the rough endoplasmic reticulum. The product, proalbumin, is in turn cleaved in the Golgi vesicles to produce the secreted albumin. Defects in ALB are a cause of familial dysalbuminemic hyperthyroxinemia (fdh). FDH is a form of euthyroid hyperthyroxinemia that is due to increased affinity of ALB for T(4). It is the most common cause of inherited euthyroid hyperthyroxinemia in caucasian population. Defects in ALB might be a cause of hyperzincemia. A variant structure of albumin could, in fact, lead to increased binding of zinc resulting in an asymptomatic augmentation of zinc concentration in the blood. Albumin has been linked to diseases as set forth by the term “Albumin-related disease”.

There is an interest to develop drugs related to Albumin. For example, Mitsubishi Pharma has pre-registered a recombinant serum albumin for Hematological disease, Renal disease and Wound healing Hemophilia. Pharming Group NV tried to develop human serum albumin for Anemia Bleeding.

An “Albumin-related disease” is selected from Microalbuminuria, albuminuria, diabetic nephropathy, hypoalbuminemia, kidney disease, proteinuria, insulin-dependent diabetes mellitus, malnutrition, non-insulin-dependent diabetes mellitus, retinal disease, nephrotic syndrome, ascites, chronic kidney failure, hypoproteinemia, kidney failure, cirrhosis, diabetes mellitus, protein-energy malnutrition, liver cirrhosis, hepatorenal syndrome, essential hypertension, kwashiorkor, cardiovascular disease, bisalbuminemia, bronchoalveolar lavage fluid, ovarian hyperstimulation syndrome, hyperthyroxinemia, liver disease, diabetic retinopathy, inflammation, protein-losing enteropathy, hypovolemia, bacterial peritonitis, liver failure, diabetic microangiopathy, hepatopulmonary syndrome, chronic liver disease, primary carcinoma of the liver cells, uremia, edema, focal segmental glomerulosclerosis 1, kernicterus, severe malnutrition, lipoid nephrosis, hepatic encephalopathy, marasmus, anemia, liver dysfunction, peripheral vascular disease, hypervolemia, autonomic neuropathy, hypertensive renal disease, acute liver failure, glomerulosclerosis, hyperglycemia, atherosclerosis, alcoholic liver cirrhosis, cyclic edema, primary biliary cirrhosis, glomerulonephritis, liver fibrosis, abnormal kidney function, insulin resistance, peritonitis, nephrosis, hypocalcemia, systemic infection, hyperbilirubinemia, chronic glomerulonephritis, dyslipidemia, metabolic acidosis, hyperhomocysteinemia, left ventricular hypertrophy, arterial hypertension, diabetic glomerulosclerosis, chronic hepatitis, encephalopathy, portal hypertension, hemolysis, allergic rhinitis, systolic hypertension, pulmonary edema, hyponatremia, hypotension, glycosuria, coronary disease, hypertensive retinopathy, hypocholesteremia, proximal renal tubular dysfunction, hypomagnesemia, alcoholic liver disease, secondary hyperparathyroidism, idiopathic membranous nephropathy, icterus, analbuminemia, familial dysalbuminemic hyperthyroxinemia, hyperzincemia, hematological disease, renal disease, wound healing Hemophilia, or bleeding anemia.

The term “treatment” as used herein encompasses any attenuation, reduction, or partial, substantial or complete prevention or blockage of disease formation, development, progression or of the formation, development or progression of any one or several or all of the symptoms of the disease.

The term “INSP052” herein refers to an INSP052 polypeptide or to an INSP052 nucleic acid molecule.

An “INSP052 polypeptide” may refer to:

The polypeptide having the sequence recited in SEQ ID NO:2 is referred to hereafter as “the INSP052 exon 1 polypeptide”. The polypeptide having the sequence recited in SEQ ID NO:4 is referred to hereafter as “the INSP052 exon 2 polypeptide”. The polypeptide having the sequence recited in SEQ ID NO:6 is referred to hereafter as “the INSP052 exon 3 polypeptide”. The polypeptide having the sequence recited in SEQ ID NO:8 is referred to hereafter as “the INSP052 exon 4 polypeptide”. The polypeptide having the sequence recited in SEQ ID NO:10 is referred to hereafter as “the INSP052 exon 5 polypeptide”. The polypeptide having the sequence recited in SEQ ID NO:12 is referred to hereafter as “the INSP052 exon 6 polypeptide”. The polypeptide having the sequence recited in SEQ ID NO:14 is referred to hereafter as “the INSP052 exon 7 polypeptide”. Combining SEQ ID NO:2, SEQ ID NO:4, SEQ ID NO:6, SEQ ID NO:8, SEQ ID NO:10, SEQ ID NO:12 and SEQ ID NO:14 produces the sequence recited in SEQ ID NO:16. The polypeptide having the sequence recited in SEQ ID NO:16 is referred to hereafter as the INSP052 polypeptide. The polypeptide having the sequence recited in SEQ ID NO:20 is the extracellular domain of INSP052. The polypeptide having the sequence recited in SEQ ID NO:22 is referred to hereafter as the extracellular domain of the mature INSP052 polypeptide. The polypeptide having the sequence recited in SEQ ID NO:24 is referred to hereafter as the mature INSP052 exon 2 polypeptide. The polypeptide having the sequence recited in SEQ ID NO:26 is referred to hereafter as the mature INSP052 polypeptide. The polypeptide having the sequence recited in SEQ ID NO:29 is referred to hereafter as the histidine-tagged, extracellular domain of mature INSP052. The polypeptide having the sequence recited in SEQ ID NO:30 is referred to hereafter as the Fc fusion of the extracellular domain of mature INSP052. The polypeptide having the sequence recited in SEQ ID NO:31 is referred to hereafter as the Ig domain containing fragment of INSP052 (INSP052Ig2). The polypeptide having the sequence recited in SEQ ID NO:32 is referred to hereafter as the extracellular INSP052 lacking the first Ig domain (INSP052-EC-DEL IG1). The polypeptide having the sequence recited in SEQ ID NO:33 is referred to hereafter as the extracellular INSP052 lacking the second Ig domain (INSP052-EC-DEL IG2). A “soluble INSP052” or “soluble form” herein refers to SEQ ID NO: 20, SEQ ID NO: 22, SEQ ID NO: 27, SEQ ID NO; 28, SEQ ID NO: 29, SEQ ID NO: 30, SEQ ID NO: 31, SEQ ID NO: 32 and/or SEQ ID NO: 33.

In one embodiment, the INSP052 polypeptide according to this embodiment consists of the amino acid sequence recited in SEQ ID NO:16 (the INSP052 polypeptide) or is a fragment of or functional equivalent thereof. In another embodiment, the INSP052 polypeptide consists of the amino acid sequence recited in any one of SEQ ID NO:2, SEQ ID NO:4, SEQ ID NO: 6, SEQ ID NO:8, SEQ ID NO:10, SEQ ID NO:12, or SEQ ID NO:14, or is a variant thereof.

The amino acid sequence recited in SEQ ID NO:20 represents the extracellular domain of INSP052 and corresponds to amino acids 1-240 of the full length protein. SEQ ID NO:22 represents the extracellular domain of mature INSP052.

As indicated in WO 03/93316, the INSP052 full length prediction encodes a type I membrane protein of 416 amino acids, related to the VEGF/PDGF receptors, belonging to the immunoglobulin superfamily. The putative signal sequence consists of amino acids 1-33 of INSP052. The Ig-like domain consists of amino acids 48 to 124. The Ig domain consists of amino acids 161 to 216. The predicted transmembrane (TM) domain consists of amino acids 241-263 of INSP052. Thus the mature extracellular domain of INSP052 consists of amino acids 34-240 of INSP052. This latter sequence is similar to two sequences disclosed in the literature as SEQIDNO434 and SEQIDNO880 (WO 04/009834; SEQ ID NO: 27 and 28). The intracellular domain of membrane-bound INSP052 corresponds to amino acids 264 to 416.

It is considered highly likely that the extracellular domain and or “soluble form” of INSP052 will fold correctly and show biological activity if additional residues C terminal and/or N terminal of these boundaries in the polypeptide sequence are included in the polypeptide fragment. In one embodiment, the INSP052 polypeptide includes additional residues C terminal and/or N terminal. For example, an additional 5, 10, 20, 30, 40, 50 or even 100 amino acid residues from the INSP052 polypeptide sequence, or from a homologous sequence, may be included at either or both the C terminal and/or N terminal of the boundaries of the receptor binding domain, without prejudicing the ability of the polypeptide fragment to fold correctly and exhibit biological activity. Extensions as large as 100 or 200 residues may be necessary due to the presence of large loops between secondary structural elements.

In one embodiment, for truncated variants of the INSP052 extracellular domain, one or a few amino acid residues (for example, 2, 3, 4, 5, 10, 15, 20, 25, 30 or more) may be deleted at either or both the C terminus or the N terminus of the domain without prejudicing biological activity.

A preferred truncated variant of the INSP052 extracellular domain may be the Ig domain containing fragment of INSP052 (INSP052Ig2) having the sequence shown in SEQ ID NO:31.

A preferred truncated variant of the INSP052 extracellular domain may be the extracellular INSP052 lacking the first Ig domain (INSP052-EC-DEL IG1) having the sequence shown in SEQ ID NO:32.

A preferred truncated variant of the INSP052 extracellular domain may be the extracellular INSP052 lacking the second Ig domain (INSP052-EC-DEL IG2) having the sequence shown in SEQ ID NO:33.

As discussed below, in one embodiment, the polypeptides of the invention may be provided in the form of a fusion protein or as “free-standing” protein. Accordingly, one embodiment of the invention provides a polypeptide which consists of the extracellular domain of INSP052. Another embodiment of the invention provides a polypeptide which consists of INSP052 (the full length protein or the extracellular domain thereof, including the mature version and truncated variants thereof) fused with at least one other polypeptide to form a fusion protein.

The “INSP052 nucleic acid molecule” may refer to a nucleic acid which comprises or consists of the nucleic acid sequence as recited in SEQ ID NO:1 (encoding the INSP052 exon 1 polypeptide), SEQ ID NO:3 (encoding the INSP052 exon 2 polypeptide), SEQ ID NO:5 (encoding the INSP052 exon 3 polypeptide), SEQ ID NO:7 (encoding the INSP052 exon 4 polypeptide), SEQ ID NO:9 (encoding the INSP052 exon 5 polypeptide), SEQ ID NO:11 (encoding the INSP052 exon 6 polypeptide), SEQ ID NO:13 (encoding the INSP052 exon 7 polypeptide), SEQ ID NO:15 (encoding the INSP052 polypeptide), SEQ ID NO:17 (encoding the mouse virtual INSP055 polypeptide), SEQ ID NO:19 (encoding the extracellular domain of the INSP052 polypeptide), SEQ ID NO:21 (encoding the extracellular domain of the INSP052 mature polypeptide), SEQ ID NO:23 (encoding the mature INSP052 exon 2 polypeptide), SEQ ID NO:25 (encoding the mature INSP052 polypeptide) or is a redundant equivalent or fragment of any one of these sequences.

Combining the sequences recited in SEQ ID NO:1, SEQ ID NO:3, SEQ ID NO:5, SEQ ID NO:7, SEQ ID NO:9, SEQ ID NO:11 and SEQ ID NO:13 produces the sequence recited in SEQ ID NO:15.

Combining the sequences recited in SEQ ID NO:23, SEQ ID NO:5, SEQ ID NO:7, SEQ ID NO:9, SEQ ID NO:11 and SEQ ID NO:13 produces the sequence recited in SEQ ID NO:25.

In one embodiment of the invention, the nucleic acid molecule encodes a polypeptide which comprises or consists of the extracellular domain of INSP052 (SEQ ID NO:20). Preferably, the nucleic acid molecule comprises or consists of the nucleic acid sequence set forth in SEQ ID NO:19. This is also set out in FIG. 7 of co-pending patent application WO03/093316, although these sequences include histidine residues added to the C terminal.

In one embodiment of the invention, the nucleic acid molecule encodes a polypeptide which comprises or consists of the extracellular domain of mature INSP052 (SEQ ID NO:22). Preferably, the nucleic acid molecule comprises or consists of the nucleic acid sequence set forth in SEQ ID NO:21. This is also set out in FIG. 7 of co-pending patent application WO03/093316, although these sequences include histidine residues added to the C terminal.

In one embodiment of the invention, the nucleic acid molecule encodes a polypeptide which comprises of consists of the variant of the extracellular domain of mature INSP052 which is the Ig-domain containing fragment of INSP052 (SEQ ID NO:31).

In one embodiment of the invention, the nucleic acid molecule encodes a polypeptide which comprises of consists of the variant of the extracellular domain of mature INSP052 which is the extracellular INSP052 lacking the first Ig domain (INSP052-EC-DEL IG1) having the sequence shown in SEQ ID NO:32.

In one embodiment of the invention, the nucleic acid molecule encodes a polypeptide which comprises of consists of the variant of the extracellular domain of mature INSP052 which is the extracellular INSP052 lacking the second Ig domain (INSP052-EC-DEL IG2) having the sequence shown in SEQ ID NO:33.

In one embodiment, the term “INSP052 polypeptide” may relate to a protein comprising all, or a portion of the sequence of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 27, SEQ ID NO: 28, SEQ ID NO: 29, SEQ ID NO: 30, SEQ ID NO: 31, SEQ ID NO: 32, or SEQ ID NO: 36 (all human except for SEQ ID NO: 18) of the enclosed sequence listing, as well as to salts, isoforms, muteins, active fractions, functional derivatives and circularly permutated derivatives thereof. In one embodiment, INSP052 from species other than human, such as mouse (e.g. SEQ ID NO: 18) or rat, may be used in accordance with the present invention, as long as there is a sufficient identity between the proteins as to allow the protein to exhibit its biological activity, and without eliciting a substantial immune response in a human being.

In one embodiment, the term “INSP052 polypeptide” may further relate to any fragment, portion, domain or sub-domain of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 27, SEQ ID NO: 28, SEQ ID NO: 29, SEQ ID NO: 30, SEQ ID NO: 31, SEQ ID NO: 32, or SEQ ID NO: 36 (all human except for SEQ ID NO: 18) showing the desired activity in infectious disease, properdin-related disease, MBL2-related disease, MASP1-related disease, MASP2-related disease, Antithrombin III-related disease, Complement factor H-related disease and/or Albumin-related disease. In one embodiment, protein fragments, isoforms, differentially glycosylated or sialylated forms or one or more domains of the protein may be used according to the invention, as long as they exhibit any beneficial effect on infectious disease, properdin-related disease, MBL2-related disease, MASP1-related disease, MASP2-related disease, Antithrombin III-related disease, Complement factor H-related disease and/or Albumin-related disease, preferably an effect which is at least comparable of the full length protein. The beneficial effect can be measured in any in vitro or in vivo tests described in the literature adequate to demonstrate an effect in infectious disease, properdin-related disease, MBL2-related disease, MASP1-related disease, MASP2-related disease, Antithrombin III-related disease, Complement factor H-related disease and/or Albumin-related disease. The biological activity of INSP052 can e.g. be measured by assaying INSP052 in its capacity to reduce infection, for example of Neisseria species, or to prevent blood clotting or platelet aggregation, or to act on the dilatation or constriction of blood vessels, or to modulate blood pressure, blood coagulation, fluid retention, opsonization, complement activation or osmotic pressure.

In one embodiment, INSP052 may be a naturally occurring, i.e. native protein, or a recombinant protein. Recombinant production may be carried out in eukaryotic cells, such as yeast cells or mammalian cells, preferably in CHO cells, HEK cells (human embryonic kidney cells) or in human fibroblast cells or cell lines. It may further be produced in prokaryotic cells such as E. coli.

Preferably, INSP052 is glycosylated at one or more sites. It may also be unglycosylated, depending on the given needs and the source of production or isolation of the protein.

Preferably, the polypeptides of the invention are glycosylated at residues 2, 71, 105, 134, 139 and/or 156 of SEQ ID NO: 22 (SEQ ID NO: 22 is taken as a reference for the residues' numbering).

The term “salts” herein refers to both salts of carboxyl groups and to acid addition salts of amino groups of INSP052 molecule or analogs thereof. Salts of a carboxyl group may be formed by means known in the art and include inorganic salts, for example, sodium, calcium, ammonium, ferric or zinc salts, and the like, and salts with organic bases as those formed, for example, with amines, such as triethanolamine, arginine or lysine, piperidine, procaine and the like. Acid addition salts include, for example, salts with mineral acids, such as, for example, hydrochloric acid or sulfuric acid, and salts with organic acids, such as, for example, acetic acid or oxalic acid. Of course, any such salts must retain the biological activity of INSP052 relevant to the present invention, i.e., exert a beneficial effect on infectious disease, properdin-related disease, MBL2-related disease, MASP1-related disease, MASP2-related disease, Antithrombin III-related disease, Complement factor H-related disease and/or Albumin-related disease.

In one embodiment, isoforms or splice variants of INSP052 may also be used according to the invention, as long as they are capable of inhibiting disease progression and/or symptoms of that disease.

In one embodiment, the term “muteins” refers to analogs of INSP052, in which one or more of the amino acid residues of natural INSP052 are replaced by different amino acid residues, or are deleted, or one or more amino acid residues are added to the natural sequence of INSP052, having preferably at least the same activity as wild type INSP052 or even having a much more potent activity. The biological activity of INSP052 can e.g. be measured by assaying INSP052 in its capacity to reduce infection, for example of Neisseria species, or to prevent blood clotting or platelet aggregation, or to act on the dilatation or constriction of blood vessels, or to modulate blood pressure, blood coagulation, fluid retention, opsonization, complement activation or osmotic pressure. Assays for assessing protein-protein interactions are well known by the person skilled in the art. Examples for such assays are ELISA type binding assays, immuno-precipitation assays, or measurement in any other suitable system such as the BIAcore system. These muteins are prepared by known synthesis and/or by site-directed mutagenesis techniques, or any other known technique suitable therefor.

Preferably, such mutein has a sequence of amino acids sufficiently duplicative of SEQ ID NO: 22, such as to have at least a substantially similar activity of SEQ ID NO: 22. The activity of an INSP052 mutant can further be tested by assaying INSP052 in its capacity to reduce infection, for example of Neisseria species, or to prevent blood clotting or platelet aggregation, or to act on the dilatation or constriction of blood vessels, or to modulate blood pressure, blood coagulation, fluid retention, opsonization, complement activation or osmotic pressure.

In one embodiment, muteins in accordance with the present invention include proteins encoded by a nucleic acid, such as DNA or RNA, which hybridizes to DNA or RNA, which encodes INSP052, in accordance with the present invention, under stringent conditions. The term “stringent conditions” refers to hybridization and subsequent washing conditions, which those of ordinary skill in the art conventionally refer to as “stringent”. See Ausubel et al., Current Protocols in Molecular Biology, supra, Interscience, N.Y., §§6.3 and 6.4 (1987, 1992), and Sambrook et al. (Sambrook, J. C., Fritsch, E. F., and Maniatis, T. (1989) Molecular Cloning: A Laboratory Manual, Cold Spring Harbor Laboratory Press, Cold Spring Harbor, N.Y.).

Without limitation, examples of stringent conditions include washing conditions 12-20° C. below the calculated Tm of the hybrid under study in, e.g., 2×SSC and 0.5% SDS for 5 minutes, 2×SSC and 0.1% SDS for 15 minutes; 0.1×SSC and 0.5% SDS at 37° C. for 30-60 minutes and then, a 0.1×SSC and 0.5% SDS at 68° C. for 30-60 minutes. Those of ordinary skill in this art understand that stringency conditions also depend on the length of the DNA sequences, oligonucleotide probes (such as 10-40 bases) or mixed oligonucleotide probes. If mixed probes are used, it is preferable to use tetramethyl ammonium chloride (TMAC) instead of SSC. See Ausubel, supra.

Preferably, any such mutein has a sequence of amino acids sufficiently duplicative of that of SEQ ID NO: 22, such as to have substantially similar, or even better, biological activity as SEQ ID NO: 22.

In a preferred embodiment, any such mutein has at least 40% identity or homology with the sequence of SEQ ID NO: 16. More preferably, it has at least 50%, at least 60%, at least 70%, at least 80% or, most preferably, at least 90% identity or homology thereto.

Identity reflects a relationship between two or more polypeptide sequences or two or more polynucleotide sequences, determined by comparing the sequences. In general, identity refers to an exact nucleotide to nucleotide or amino acid to amino acid correspondence of the two polynucleotides or two polypeptide sequences, respectively, over the length of the sequences being compared.

For sequences where there is not an exact correspondence, a “% identity” may be determined. In general, the two sequences to be compared are aligned to give a maximum correlation between the sequences. This may include inserting “gaps” in either one or both sequences, to enhance the degree of alignment. A % identity may be determined over the whole length of each of the sequences being compared (so-called global alignment), that is particularly suitable for sequences of the same or very similar length, or over shorter, defined lengths (so-called local alignment), that is more suitable for sequences of unequal length.

Methods for comparing the identity and homology of two or more sequences are well known in the art. Thus for instance, programs available in the Wisconsin Sequence Analysis Package, version 9.1 (Devereux J et al, 1984), for example the programs BESTFIT and GAP, may be used to determine the % identity between two polynucleotides and the % identity and the % homology between two polypeptide sequences. BESTFIT uses the “local homology” algorithm of Smith and Waterman (1981) and finds the best single region of similarity between two sequences. Other programs for determining identity and/or similarity between sequences are also known in the art, for instance the BLAST family of programs (Altschul S F et al, 1990, Altschul S F et al, 1997, accessible through the home page of the NCBI at www.ncbi.nlm.nih.gov) and FASTA (Pearson W R, 1990; Pearson 1988).

Muteins of INSP052, which can be used in accordance with the present invention, or nucleic acids encoding them, include a finite set of substantially corresponding sequences as substitution peptides or polynucleotides which can be routinely obtained by one of ordinary skill in the art, without undue experimentation, based on the teachings and guidance presented herein.

In one embodiment, preferred changes for muteins in accordance with the present invention are what are known as “conservative” substitutions. Conservative amino acid substitutions of INSP052 polypeptides or proteins, may include synonymous amino acids within a group which have sufficiently similar physicochemical properties that substitution between members of the group will preserve the biological function of the molecule (Grantham, 1974). It is clear that insertions and deletions of amino acids may also be made in the above-defined sequences without altering their function, particularly if the insertions or deletions only involve a few amino acids, e.g., under thirty, and preferably under ten, and do not remove or displace amino acids which are critical to a functional conformation, e.g., cysteine residues. Proteins and muteins produced by such deletions and/or insertions come within the purview of the present invention.

Preferably, the synonymous amino acid groups are those defined in Table I. More preferably, the synonymous amino acid groups are those defined in Table II; and most preferably the synonymous amino acid groups are those defined in Table III.

TABLE I
Preferred Groups of Synonymous Amino Acids

| Amino Acid | Synonymous Group |
|---|---|
| Ser | Ser, Thr, Gly, Asn |
| Arg | Arg, Gln, Lys, Glu, His |
| Leu | Ile, Phe, Tyr, Met, Val, Leu |
| Pro | Gly, Ala, Thr, Pro |
| Thr | Pro, Ser, Ala, Gly, His, Gln, Thr |
| Ala | Gly, Thr, Pro, Ala |
| Val | Met, Tyr, Phe, Ile, Leu, Val |
| Gly | Ala, Thr, Pro, Ser, Gly |
| Ile | Met, Tyr, Phe, Val, Leu, Ile |
| Phe | Trp, Met, Tyr, Ile, Val, Leu, Phe |
| Tyr | Trp, Met, Phe, Ile, Val, Leu, Tyr |
| Cys | Ser, Thr, Cys |
| His | Glu, Lys, Gln, Thr, Arg, His |
| Gln | Glu, Lys, Asn, His, Thr, Arg, Gln |
| Asn | Gln, Asp, Ser, Asn |
| Lys | Glu, Gln, His, Arg, Lys |
| Asp | Glu, Asn, Asp |
| Glu | Asp, Lys, Asn, Gln, His, Arg, Glu |
| Met | Phe, Ile, Val, Leu, Met |
| Trp | Trp |

TABLE II
More Preferred Groups of Synonymous Amino Acids

| Amino Acid | Synonymous Group |
|---|---|
| Ser | Ser |
| Arg | His, Lys, Arg |
| Leu | Leu, Ile, Phe, Met |
| Pro | Ala, Pro |
| Thr | Thr |
| Ala | Pro, Ala |
| Val | Val, Met, Ile |
| Gly | Gly |
| Ile | Ile, Met, Phe, Val, Leu |
| Phe | Met, Tyr, Ile, Leu, Phe |
| Tyr | Phe, Tyr |
| Cys | Cys, Ser |
| His | His, Gln, Arg |
| Gln | Glu, Gln, His |
| Asn | Asp, Asn |
| Lys | Lys, Arg |
| Asp | Asp, Asn |
| Glu | Glu, Gln |
| Met | Met, Phe, Ile, Val, Leu |
| Trp | Trp |

TABLE III
Most Preferred Groups of Synonymous Amino Acids

| Amino Acid | Synonymous Group |
|---|---|
| Ser | Ser |
| Arg | Arg |
| Leu | Leu, Ile, Met |
| Pro | Pro |
| Thr | Thr |
| Ala | Ala |
| Val | Val |
| Gly | Gly |
| Ile | Ile, Met, Leu |
| Phe | Phe |
| Tyr | Tyr |
| Cys | Cys, Ser |
| His | His |
| Gln | Gln |
| Asn | Asn |
| Lys | Lys |
| Asp | Asp |
| Glu | Glu |
| Met | Met, Ile, Leu |
| Trp | Met |

Examples of production of amino acid substitutions in proteins which can be used for obtaining muteins of INSP052 polypeptides or proteins, for use in the present invention include any known method steps, such as presented in U.S. Pat. Nos. 4,959,314, 4,588,585 and 4,737,462, to Mark et al; U.S. Pat. No. 5,116,943 to Koths et al., U.S. Pat. No. 4,965,195 to Namen et al; U.S. Pat. No. 4,879,111 to Chong et al; and U.S. Pat. No. 5,017,691 to Lee et al; and lysine substituted proteins presented in U.S. Pat. No. 4,904,584 (Shaw et al).

The term “fusion protein” refers to a polypeptide comprising INSP052, or a mutein thereof, fused with another protein, which, e.g., has an extended residence time in body fluids. Fusion proteins comprising all or a functional part of INSP052 fused to all or a functional part of a protein capable of improving the biological activities of the molecule, like half-life in the human body, for instance, are preferred according to the invention. In a preferred embodiment the fusion protein comprises an immunoglobulin (Ig) fusion. Fusion proteins comprising all or part of INSP052 fused to all or part of an immunoglobulin are highly preferred. They can be monomeric or multimeric, hetero- or homomultimeric. Advantageously, the fusion protein comprises the constant region of an immunoglobulin, in particular of the Fc portion of the immunoglobulin. Embodiments in which the immunoglobulin is of the IgG1 or IgG2 isotype are further preferred according to the invention. Preferably, the fusion is an Fc fusion. Preferably, the fusion protein consists of SEQ ID NO: 30.

INSP052 may thus be fused to another protein, polypeptide or the like, e.g., an immunoglobulin or a fragment thereof. The fusion may be direct, or via a short linker peptide which can be as short as 1 to 3 amino acid residues in length or longer, for example, 13 amino acid residues in length. Said linker may be a tripeptide of the sequence E-F-M (Glu-Phe-Met), for example, or a 13-amino acid linker sequence comprising Glu-Phe-Gly-Ala-Gly-Leu-Val-Leu-Gly-Gly-Gln-Phe-Met introduced between the INSP052 sequence and the immunoglobulin sequence.

“Functional derivatives” as used herein cover derivatives of INSP052, and their muteins and fusion proteins, which may be prepared from the functional groups which occur as side chains on the residues or the N— or C-terminal groups, by means known in the art, and are included in the invention as long as they remain pharmaceutically acceptable, i.e. they do not destroy the activity of the protein which is at least substantially similar to the activity of SEQ ID NO: 22, and do not confer toxic properties on compositions containing it. Therefore, in a preferred embodiment the functional derivative comprises at least one moiety attached to one or more functional groups, which occur as one or more side chains on the amino acid residues.

In accordance with the present invention, polyethylene glycol (PEG) side-chains are highly preferred moieties. PEG side chains may mask antigenic sites and extend the residence of the substance they are attached to in body fluids. Other derivatives include aliphatic esters of the carboxyl groups, amides of the carboxyl groups by reaction with ammonia or with primary or secondary amines, N-acyl derivatives of free amino groups of the amino acid residues formed with acyl moieties (e.g. alkanoyl or carbocyclic aroyl groups) or O-acyl derivatives of free hydroxyl groups (for example that of seryl or threonyl residues) formed with acyl moieties.

“Active fractions” of INSP052 and its muteins and fusion proteins, cover any fragment or precursors of the polypeptide chain of the protein molecule alone or together with associated molecules or residues linked thereto, e.g., sugar or phosphate residues, or aggregates of the protein molecule or the sugar residues by themselves, provided said active fraction has at least a substantially similar activity to SEQ ID NO: 22.

In accordance with the present invention, INSP052 may also be administered to the human body in form of a vector comprising said nucleic acid molecule. Therefore, the invention further relates to the use of a vector comprising said nucleic acid molecule for the manufacture of a medicament for the treatment and/or prevention of infectious disease, properdin-related disease, MBL2-related disease, MASP1-related disease, MASP2-related disease, Antithrombin III-related disease, Complement factor H-related disease and/or Albumin-related disease. Preferably, the vector is an expression vector, comprising a promoter operably linked to all or part of the coding sequence of INSP052. In a further preferred embodiment, the vector is a gene therapy vector. Gene therapy vectors are known in the art, most of them are virally derived vectors, such as adenoviral or lentiviral vectors.

According to the invention, INSP052 may also be administered to the human body in form of a cell producing and/or secreting INSP052. Therefore, the invention further relates to the use of a cell expressing INSP052 for the manufacture of a medicament for the treatment and/or prevention of infectious disease, properdin-related disease, MBL2-related disease, MASP1-related disease, MASP2-related disease, Antithrombin III-related disease, Complement factor H-related disease and/or Albumin-related disease, i.e. to cell therapy for the treatment and/or prevention of infectious disease, properdin-related disease, MBL2-related disease, MASP1-related disease, MASP2-related disease, Antithrombin III-related disease, Complement factor H-related disease and/or Albumin-related disease. The cell may be a naturally producing INSP052 and/or a transfected cell that produces recombinant INSP052. Preferred are cells expressing and secreting high amounts of the protein, such as over-expressing cells carrying high copy numbers of an expression vector comprising a nucleic acid molecule encoding INSP052.

The invention further relates to a cell comprising a vector comprising a nucleic acid molecule encoding all or part of INSP052 for the preparation of a medicament for treatment and/or prevention of infectious disease, properdin-related disease, MBL2-related disease, MASP1-related disease, MASP2-related disease, Antithrombin III-related disease, Complement factor H-related disease and/or Albumin-related disease. A cell that has been genetically modified to produce a polypeptide according to the invention is also within the scope of the present invention.

The use of an expression vector for inducing and/or enhancing the endogenous production of INSP052 in a cell normally silent or expressing amounts of the inhibitor which are not sufficient, are also contemplated according to the invention. Thus, the invention makes use of a technology known as endogenous gene activation (EGA) for the production of the desired protein.

According to the invention, INSP052 can be administered alone or in combination with several other therapeutic regimens or agents (e.g. multiple drug regimens) to obtain an additive or synergistic effect for the treatment and/or prevention of infectious disease, properdin-related disease, MBL2-related disease, MASP1-related disease, MASP2-related disease, Antithrombin III-related disease, Complement factor H-related disease and/or Albumin-related disease. Therefore, preferably, the medicament of the invention further comprises:
Interferon, in particular interferon-α, interferon-β, or interferon-γ, or
A Tumor Necrosis Factor (TNF) antagonist, in particular soluble TNFRs, such as soluble p55 (TBPI) and/or soluble p75 (TBP II), or
An anti-infectious agent, or
An anti-blood clotting agent.

The anti-blood clotting agent may be selected from Nitrates, nitroglycerin, isosorbide dinitrate, isosorbide mononitrate, vitamin C or E, Beta-blockers, propranolol (Inderal, Ciplar), labetalol (Normadate), acebutolol (Sectral), atenolol (Aten, Tenormin, Betacard, Tensimin), metoprolol (Betaloc, Metolar), bisoprolol (Concor), Carvedilol (Carvedil, Carloc, Carvil), anticoagulants, heparin, warfarin, anti-platelet drug, aspirin, glycoprotein IIb/IIIa receptor antagonists, clopidogrel, NSAIDs, Enoxaparin (Clexane), dalteparin (Fragmin), reviparin (Clivarine), abciximab (ReoPro, Centocor), eptifibatide, lamifiban, tirofiban, abciximab, Clopidogrel (Deplatt, Clopilet), ticlopidine (Tyklid, Ticlop), Hirudin, Bivalirudin, argatroban, danaparoid, Statins, Angiotensin Converting Enzyme Inhibitors Angiotensin converting enzyme (ACE) inhibitors, ramipril (Cardace), captopril (Capotril, Aceten), enalapril (Envas, Vasonorm), lisinopril (Prevace, Lipril, Zestril), fosinopril, Calcium Channel Blockers, verapamil (Calan, Isoptin), nifedipine (Adalat, Depin), nicardipine (Cardene), amlodipine (Amdepin, Corvadil), diltiazem (Cardizem, Dilzem), bepridil (Vascor), Ranolazine, Nicorandil, Antibiotics, tetracyclines, quinolones, Folic acid, Thrombolytics, recombinant tissue plasminogen activators (rt-Pas), alteplase (Activase and reteplase (Retavase)), Fibrin-Depleting Agent, ancrod, batroxobin (Defibrase), Thienopyridines, Clopidogrel (Plavix), ticlopidine (Ticlid), thienopyridines, (Clopidogrel), Direct Thrombin Inhibitors (DTIs), hirudin, argatroban (Novastan), bivalirudin (Angiomax), danaproid (Orgaran), lepirudin (Refludan), desirudin (Revasc), inogatran, efegatran, Ximelagatran (Exanta), antifibrinolytics, tranexamic acid, or epsilon amino-caproic acid.

The anti-infectious agent may be selected from pentamidine, antibiotics, colistine, aminoglycosides or amphotericin B.

All treatments are intended for simultaneous, sequential or separate use.

Pharmaceutical compositions comprising one or more of the above substances, together with INSP052, are within the scope of the present invention.

In yet a further embodiment of the invention, INSP052 is used in combination with an interferon. Preferably, the interferon is selected among interferon-α, interferon-β, or interferon-γ. Interferons have also been associated to infectious disease, properdin-related disease, MBL2-related disease, MASP1-related disease, MASP2-related disease, Antithrombin III-related disease, Complement factor H-related disease and/or Albumin-related disease. For example, interferon-α has been involved in neutropenia, thrombocytopenia, hepatitis C or hepatitis B. For example, interferon-β has been involved in hepatitis B, hepatitis C, virus diseases, hiv-infections and influenza. For example, interferon-γ has been associated with Tuberculosis, virus diseases, visceral or cutaneous leishmaniasis, hiv infections, hepatitis B, hepatitis C, influenza, diabetes, malaria, bacterial infections, mycobacterium infections and filarial elephantiasis.

In yet a further embodiment of the invention, INSP052 is used in combination with a TNF antagonist. TNF antagonists exert their activity in several ways. First, antagonists can bind to or sequester the TNF molecule itself with sufficient affinity and specificity to partially or substantially neutralise the TNF epitope or epitopes responsible for TNF receptor binding (hereinafter termed “sequestering antagonists”). A sequestering antagonist may be, for example, an antibody directed against TNF. For example, TNF-α has been involved in virus diseases, malaria, septic shock, bacterial infections, mycobacterium infections, bacterial meningitis, hiv infections and tuberculosis.

Alternatively, TNF antagonists can inhibit the TNF signalling pathway activated by the cell surface receptor after TNF binding (hereinafter termed “signalling antagonists”). TNF antagonists are easily identified and evaluated by routine screening of candidates for their effect on the activity of native TNF on susceptible cell lines in vitro, for example human B cells, in which TNF causes proliferation and immunoglobulin secretion. The assay contains TNF formulation at varying dilutions of candidate antagonist, e.g. from 0.1 to 100 times the molar amount of TNF used in the assay, and controls with no TNF or only antagonist (Tucci et al., 1992).

Sequestering antagonists are the preferred TNF antagonists to be used according to the present invention. Amongst sequestering antagonists, those polypeptides that bind TNF with high affinity and possess low immunogenicity are preferred. Soluble TNF receptor molecules and neutralising antibodies to TNF are particularly preferred. For example, soluble forms of TNF-RI (p55) and TNF-RII (p75) are useful in the present invention. Truncated forms of these receptors, comprising the extracellular domains of the receptors or functional portions thereof, are more particularly preferred antagonists according to the present invention. Truncated soluble TNF type-I and type-II receptors are described in EP914431, for example.

Truncated forms of the TNF receptors are soluble and have been detected in urine and serum as about 30 kDa or 40 kDa TNF inhibitory binding proteins, which are called TBPI and TBPII, respectively (Engelmann et al., 1990). The simultaneous, sequential, or separate use of INSP052 with the TNF antagonist and/or an Interferon is preferred, according to the invention.

According to the invention, TBPI and TBPII are preferred TNF antagonists to be used in combination with INSP052. Derivatives, fragments, regions and biologically active portions of the receptor molecules functionally resemble the receptor molecules that can also be used in the present invention. Such biologically active equivalent or derivative of the receptor molecule refers to the portion of the polypeptide, or of the sequence encoding the receptor molecule, that is of sufficient size and able to bind TNF with such an affinity that the interaction with the membrane-bound TNF receptor is inhibited or blocked.

In a further preferred embodiment, human soluble TNF-RI (TBPI) is the TNF antagonist to be used according to the invention. The natural and recombinant soluble TNF receptor molecules and methods of their production have been described in the European Patents EP 308 378, EP 398 327 and EP 433 900.

Whilst it may be beneficial to block TNF-α in early stages of the disease, it has been discussed that in later stages, TNF itself may exert a beneficial effect on disease progression (Abraham et al., 2000). Therefore, the invention further relates to a combination of INSP052 and TNF for treatment or prevention of infectious disease, properdin-related disease, MBL2-related disease, MASP1-related disease, MASP2-related disease, Antithrombin III-related disease, Complement factor H-related disease and/or Albumin-related disease, in particular in advanced stages of disease. TNF-α or TNF-β may be used in accordance with the invention.

The invention further relates to a pharmaceutical composition comprising INSP052, optionally together with one or more pharmaceutically acceptable carriers, diluents or excipients, for the treatment and/or prevention of infectious disease, properdin-related disease, MBL2-related disease, MASP1-related disease, MASP2-related disease, Antithrombin III-related disease, Complement factor H-related disease and/or Albumin-related disease. The pharmaceutical composition may further comprise any of the above-identified further components, and preferably an interferon.

The pharmaceutical composition according to the invention may also comprise a vector comprising a nucleic acid molecule according to the invention, or a cell expressing INSP052.

The active ingredients of the pharmaceutical, i.e. polypeptides, nucleic acids or cells according to the invention, or combinations thereof, as well as the combinations of substances mentioned above, may be administered to an individual in a variety of ways. The routes of administration include intradermal, transdermal (e.g. in slow release formulations), intramuscular, intraperitoneal, intravenous, subcutaneous, oral, epidural, topical, and intranasal routes. Any other therapeutically efficacious route of administration can be used, for example absorption through epithelial or endothelial tissues or by gene therapy wherein a DNA molecule encoding the active agent is administered to the patient (e.g. via a vector), which causes the active agent to be expressed and secreted in vivo. In addition, the protein(s) according to the invention can be administered together with other components of biologically active agents such as pharmaceutically acceptable surfactants, excipients, carriers, diluents and vehicles.

The definition of “pharmaceutically acceptable” is meant to encompass any carrier, which does not interfere with effectiveness of the biological activity of the active ingredient and that is not toxic to the host to which it is administered. For example, for parenteral administration, the active protein(s) may be formulated in a unit dosage form for injection in vehicles such as saline, dextrose solution, serum albumin and Ringer's solution.

For parenteral (e.g. intravenous, subcutaneous, intramuscular) administration, the active protein(s) can be formulated as a solution, suspension, emulsion or lyophilised powder in association with a pharmaceutically acceptable parenteral vehicle (e.g. water, saline, dextrose solution) and additives that maintain isotonicity (e.g. mannitol) or chemical stability (e.g. preservatives and buffers). The formulation is sterilized by commonly used techniques.

The bioavailability of the active protein(s) according to the invention can also be ameliorated by using conjugation procedures which increase the half-life of the molecule in the human body, for example linking the molecule to polyethylenglycol, as described in the PCT Patent Application WO 92/13095.

The therapeutically effective amount of the active protein(s) will be a function of many variables, including the type of receptor, the affinity of the substance according to the invention to its receptor, any residual cytotoxic activity exhibited thereby, the route of administration, the clinical condition of the patient.

A “therapeutically effective amount” is such that when administered, the substance according to the invention results in a beneficial effect on disease development or progression in vivo. The dosage administered, as single or multiple doses, to an individual will vary depending upon a variety of factors, including the pharmacokinetic properties of INSP052, the route of administration, patient conditions and characteristics (sex, age, body weight, health, size), extent of symptoms, concurrent treatments, frequency of treatment and the effect desired. Adjustment and manipulation of established dosage ranges are well within the ability of those skilled in the art.

The dose of the polypeptide according to the invention required will vary from about 0.0001 to 100 mg/kg or about 0.01 to 10 mg/kg or about 0.1 to 5 mg/kg or about 1 to 3 mg/kg, although as noted above this will be subject to a great deal of therapeutic discretion. The medicament of the invention may be administered daily, every other day, or three times per week.

The daily doses are usually given in divided doses or in sustained release form effective to obtain the desired results. Second or subsequent administrations can be performed at a dosage, which is the same, less than or greater than the initial or previous dose administered to the individual. A second or subsequent administration can be administered during or prior to onset of the disease.

The invention further relates to a method for treating and/or preventing infectious disease, properdin-related disease, MBL2-related disease, MASP1-related disease, MASP2-related disease, Antithrombin III-related disease, Complement factor H-related disease and/or Albumin-related disease, comprising administering to a patient in need thereof an effective amount of a substance according to the invention, optionally together with a pharmaceutically acceptable carrier. Alternatively, or additionally, a cell producing INSP052 or a nucleic acid molecule of the invention, optionally comprised in an expression vector, may be administered according to the invention.

The expression vector may be administered systemically. Preferably the expression vector is administered by intramuscular injection. A further preferred route of administration is inhalation, in particular if the lungs are involved in the disease (e.g. infectious diseases of the lung). Topical administration of an expression vector comprising INSP052 sequences, or of an INSP052 polypeptide according to the invention, is a further preferred route of administration, in particular if there is an involvement of the skin.

The invention further relates to a method for the preparation of a pharmaceutical composition comprising admixing an effective amount of INSP052 with a pharmaceutically acceptable carrier, and to a method of treatment and/or prevention of infectious disease, properdin-related disease, MBL2-related disease, MASP1-related disease, MASP2-related disease, Antithrombin III-related disease, Complement factor H-related disease and/or Albumin-related disease comprising administering to a host in need thereof an effective amount of INSP052.

All references cited herein, including journal articles or abstracts, published or unpublished patent applications, issued patents or any other references, are entirely incorporated by reference herein, including all data, tables, figures and text presented in the cited references. Additionally, the entire contents of the references cited within the references cited herein are also entirely incorporated by reference.

Reference to known method steps, conventional methods steps, known methods or conventional methods is not any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various application such specific embodiments, without undue experimentation, without departing from the general concept of the present invention. Therefore, such adaptations and modifications are intended to be within the meaning an range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

Having now described the invention, it will be more readily understood by reference to the following examples that are provided by way of illustration and are not intended to be limiting of the present invention.

### EXAMPLES

### Example 1

### Cloning and Expression

Cloning of extracellular INSP052, construction of mammalian cell expression vectors, expression in mammalian cells and purification of extracellular INSP052 and histidine tagged version of extracellular domain are described in Examples 2 and 3 of International Application WO2003/093316. and No. PCT/GB2004/004772.

The polypeptide having the sequence recited in SEQ ID NO:30 (referred to hereafter as the Fc fusion of the extracellular domain of mature INSP052) has been purified twice (500 ml each) and 7.9 mg and 2.4 mg were recovered. The polypeptide having the sequence recited in SEQ ID NO:32 (referred to as the extracellular INSP052 lacking the first Ig domain or INSP052-EC-DEL IG1) has been purified and recovered (2 purifications (500 ml) of recovery 0.7 mg and 0.3 mg, respectively and 1 purification (3000 ml) of recovery 7 mg). The polypeptide having the sequence recited in SEQ ID NO:33 (referred to hereafter as the extracellular INSP052 lacking the second Ig domain or INSP052-EC-DEL IG2) has been purified (500 ml) and recovered at Western Blot levels.

### Example 2

### Binding Partners of INSP052

### I. Introduction

The SELDI-TOF MS technique (Surface-enhanced Laser Desorption/Ionization Time-of-flight Mass Spectrometry/ProteinChip™ Arrays) was used to identify potential binding partners of soluble INSP052 (sINSP052—corresponding to the mature extracellular part of membrane bound INSP052, SEQ ID NO: 22). Briefly, the following steps were performed:
1. Acquiring the sample from mouse,
2. SELDI analysis and optimization,
3. Scale-up to beads,
4. Analyse eluate on SELDI,
5. HPLC of bead eluate,
6. Analyse HPLC fractions on chip and gel,
7. Digestion of isolated proteins and
8. Nano LC MS/MS and identification of proteins.

### II. Material and Methods

First, intravenous LPS or NaCl (control) was injected in female CH3 mice and blood was collected through intra cardiac puncuration after 3 hours. The mice were injected i.p. with 300 μg/Kg LPS. For plasma the blood was collected in heparin treated tubes and for serum in untreated tubes. The tubes were centrifuged at 13,000 rpm for 10 min. The supernatant (serum or plasma samples) was collected and used for the experiment.

Then, 1.25 μg of the bait protein sINSP052 or control IL-18 (IL-18 as a control protein binding of IL-18BP) was applied to each spot on RS100 ProteinChip™ Arrays (Ciphergen Biosystems Inc., Fremont, Calif.) and incubated 2 h (room temperature) in a humidified chamber. Non-specific binding sites were blocked with 0.5 M ethanolamine/PBS for 1 hour (room temperature). Arrays were washed 3 times for 5 min with PBS, 0.1% Triton X-100. Plasma samples were diluted 1:1 in PBS, 0.1% Triton X-100, and incubated on the arrays (2 h, room temperature). Optimal conditions for the samples are achieved with an incubation time of 4 hours. The arrays were then washed for 2×5 min with PBS, 0.1% Triton X-100 and 1×3 min with 1M Urea/2%CHAPS/0.5M NaCl/50 mM Tris pH 9. They were rinsed with 5 mM HEPES pH 7.2 (15 sec room temperature). The matrix, sinapinic acid was applied to each spot on the array and mass analysis was performed by SELDI-TOF-MS, using the ProteinChip™ Biology System II. Spectra were generated using laser intensity 200, sensitivity 9 and 10 and mass focus between 10-40 kDa and 20-160 kDa.

Spectra of controls and samples from different chips were merged into the same file and treated identically for analysis. The spectra were calibrated using the all-in-1 protein standard from Ciphergen biosystems Inc. following manufacturers indications. For comparison between spectra, they were normalized to total ion current. Differentially expressed peaks were identified using the Ciphergen Biomarker Wizard software.

For identification of binding proteins, IDM affinity beads (Ciphergen biosystems) were used, applying a similar protocol as the one used on the RS100 chip. 1 mg bait protein (sINSP052) in 50 mM NaAcetate buffer pH 5.0 was incubated on 600 μl beads overnight at room temperature. The beads were washed 2×5 min 50 mM Na Acetate buffer pH 5.0 and blocked with 0.5M Tris-HCl pH 9.0, 0.1% Triton-X100 for 2 hours. They were washed 3×10 min 0.1% Triton-X100 in PBS and 2×10 min in PBS. Plasma samples were diluted in 1:1 in PBS, 0.1% Triton X-100, and incubated on the beads (overnight, room temperature). Washed 2×5 min with 0.2M Urea/0.1% CHAPS/0.2M NaCl/50 mM Tris pH 7.5 and 2×5 min with PBS. Interacting proteins were eluted with 50% acetonitrile, 0.3%TFA (Tri-phosphatidic acid). Eluates were examined using the NP20 protein chip. 5 μl eluate was added per spot. Spots were washed with deionised water and undiluted sinapinic acid was added. Spectra were generated using laser intensity 180/200, sensitivity 8 and mass focus between 2-30 kDa and 20-160 kDa.

The eluate was further fractionated on a C4 (butyl) column (reverse phase), with a 2.1 mm diameter (VYDAC 214TP™ Series, Grace Vydac) at a flowrate of 0.2 ml/min. The column was developed with a continuous linear acetonitrile gradient 0 to 90% in 0.1% TFA. 1 min fractions were collected. The fractions were lyophilized and the protein content determined using the NP20 protein chip.

RP-HPLC fractions containing the proteins of interest were trypsin digested for identification, using Rapigest™ SF (Waters) following manufacturers protocol.

The pooled fractions were also analysed by gel electrophoresis and silverstain.

Mass spectrometric experiments were conducted using a Q-Star quadrupole/time-of-flight instrument (MDS/Sciex). The interface was connected to an Ultimate HPLC system (LC-Packings), equipped with a capillary C-18 RP column (75 μm inner diameter, 150 mm length). Peptide digests were injected onto the column using a Famos autosampler (LC-Packings) and separated on the column by a gradient of acetonitrile in water in the presence of 0.1% formic acid at a flow rate of 200 nl/min. The column was connected to a nano-emitter and the eluent sprayed towards the orifice of the mass spectrometer using a current of 2400 V. The mass spectrometer is operated in a data dependent acquisition mode. A second survey scans are performed in the MS mode followed by 4 MS/MS scans when [M+2H]²⁺ or [M+3H]³⁺ precursor ions are detected above a signal of 20 counts/second. Tandem mass spectra were obtained using nitrogen as a target gas at collision energies that were set automatically depending on the mass and the charge of the precursor ion.

Peptide sequences were determined after transfer of the MS/MS data to the Mascot program and searches performed in protein databases available in house.

The protocols used are presented below:

### a) Fishing Protein Binding Partners Using the Ciphergen RS100 Protein Chip

1. Added 5 μl of 1 mg/ml protein diluted 1:3 in NaHCO3 to each spot

2. Incubated for 2 h in a humidified chamber.

3. Added 50 μl 0.5M ethanolamine and blocked for 1 h at RT by mixing (Micromix5 20/02/60).

4. Washed 2×5 min with 0.1% TritonX100 in PBS (micromix5 20/3/5)

5. Prepared samples and added 50 μl of plasma solution/spot
i. Plasma from LPS stimulated mice diluted 1:1 in PBS/0.1% Triton

6. Centrifuged the bioprocessor 1 min at 500 rpm. Incubated for 2 h at RT, shaking (Micromix5 20/2/120)

7. Washed
i. 3×5 min with 0.1% TritonX100 in PBS.
ii. 1×3 min with 1M Urea/2%CHAPS/0.5M NaCl/50 mM Tris pH 9

8. Removed the array from the holder and placed it in a 15 ml falcon containing 5 mM Hepes pH 7.5. (10 sec)

9. Added 2×0.5 μl SPA solution.

10. Let airdry and analyzed the samples.

11. The chips were analysed with the Ciphergen SELDI using the following reading conditions: 200-10-40-65-100-51 or 200-9-20-160-160-50 laser intensity-sensitivity-massfocus low-massfocus high-Maximum mass-mass focus)

b) Binding INSP052 to Affinity Beads and Incubating with Mouse Plasma

i) Binding the Bait Protein to Beads

1. Took out the beads and kept at RT for 30 min

2. Mixed beads for 5 min on roller

3. Added 200 μl of beads into two 1.5 ml eppendorff tubes

4. Washed 6 times with 1 ml water

5. Added 600 μl
a. 2.2 mg/ml INSP052 (13495F13P1 1.7 mg/ml concentrated to 4 mg/ml) (1320 μg total)
b. 1.95 mg/ml IL 18 (hIL 18mature, E. Coli, Batch LM10821-110 1 mg/ml concentrated to 4 mg/ml) (1170 μg total) in 50 mM Na Acetate buffer pH 5.0 and incubate overnight at RT on the roller

ii) Saturating Unbound Sites and Incubating with Plasma

6. Removed supernatant, determined protein concentration and stored (See below for protein concentration)

7. Washed 2×1 ml 50 mM NaAcetate buffer pH 5.0

8. Add 600 μl of 0.5M Tris-HCl pH 9, 0.1% TritonX100 (to deactivate possible remaining active groups) and incubate for 2 h on a roller at RT

9. Wash 3×10 min with 1 ml 0.1% TritonX100 in PBS

10. Wash 2×10 min with 1 ml PBS

11. Put 20 μl of INSP052 bound beads into eight 1.5 ml eppitubes

12. Mix 600 μl of serum+600 μl 0.1% Triton in PBS+56 μl 25×protease inhibitor (complete mix) and added to beads
a. Ctrl serum
b. LPS serum
and incubate for o/n at RT (18 hours)

iii) Washing and Eluting from the Beads

13. Prepared washing and elution solutions and filtered them

14. Removed supernatant and store at −80° C. for a possible incubation with a new set of bound beads

15. Added 2×100 μl of PBS into each eppi to transfer same samples into only one eppi, giving total of 2 eppis.

16. Washed beads 1×5 min 400 μl & 1×5 min 500 μl with 0.2M Urea/0.1% CHAPS/0.2M NaCl/50 mM Tris pH 7.5. Kept wash solution and examined with SELDI.

17. Washed beads 2×5 min with 1 ml of 1×PBS

18. Eluted with 200 μl 50% acetonitrile, 0.3% TFA (0.2 ml H2O, 0.5 ml 100% ACN, 0.3 ml 1% TFA). Vortexed gently for 30 sec, rotate for 3 min and vortexed again gently for 30 sec. Placed eluate in a separate eppi.

19. Left beads in 4C for 3 h, added 200 μl 50% acetonitrile, 0.3% TFA and redid elution procedure.

20. Added additional 200 μl 50% acetonitrile, 0.3% TFA and redid elution procedure.

21. Placed 3 μl for eluate 1 and 6 μl for eluates 2 and 3 on a NP20 spot. Let dry. Wash 2×5× up-and-down with H2O. Let dry. Add 0.8 μl 100% SPA

22. Analysed low and high molecular weight proteins with separate SELDI protocols
a. Low: 800-8-2-30-160-16
b. High: 200-8-20-160-160-50

23. Looked at the various solution with gel electrophoresis and silverstain

### c) Reverse-HPLC Fractionation of the Bead Eluate

The eluate of INSP052 bound beads, incubated with LPS plasma (from 211204) was run through a HPLC column. Using parameters:
Column:
C4 (butyl) column (reverse phase), 2.1
Solvant A: 0.1% TFA
Solvant B: 0.1% TFA, 90% acetonitrile
Wash column:
30 min 0-90% acetonitrile, 0.1% TFA, 0.2 ml/min
Inject sample:
100 μl of lyophilized sample resuspended in 0.1% TFA
followed by 5 min solvant A (0.1% TFA)
Elution:
105 min elution 0-90% acetonitrile, 0.1% TFA, 0.2 ml/min
5 min elution 90% acetonitrile, 0.1% TFA, 0.2 ml/min
10 min wash 0.1% TFA

1 min fractions were collected. Each fraction was analysed on the SELDI. When 10 μl of 200 μl was absorbed on the NP20 protein chip spot only the highest HPLC peaks could be visualized. Therefore, the fractions corresponding to the smaller peaks were lyophilized, resuspended in 15 μl 0.3% TFA, 50% acetonitrile and 5 μl was added to the chip

### d) Rapigest Protein Digestion for MALDI

1. The fractions (200 μl) were lyophilized separately

2. 50 μl was used to resuspend and merge 4 fractions. The tubes were washed further with 50 μl that was added to the previous volume.
3. The samples were lyophilised

The samples were resuspended in 50 μl 0.2 w/v Rapigest and the Rapigest protocol was followed.

### III. Results

Protein peaks of 20, 50, 53 and 56 kDa were seen to bind to array spots with immobilized sINSP052 but not to array spots with the immobilized control protein, IL-18. LPS stimulated plasma generally showed higher peaks of the binding partners (more binding) compared to the control plasma. Plasma samples gave higher peaks when incubated on immobilized sINSP052 compared to serum.

The mouse peptide sequences were thus determined after transfer of the MS/MS data to the Mascot program and searches performed in protein databases available in house. Several binding partners were identified as follows:
Properdin. The properdin size (49959 Da) fits with the observed SELDI mass of 56 kDa (the protein is glycosylated),
Mannose-binding lectin C precursor (MBL-C). The size of 25 kDa correlates with the gel band (ca 30 kDa). The protein forms tight polymers through disulfide bonds, explaining the 50.6 kDa peak on SELDI.
MUSCRARF NID or P100 serine protease of the Ra reactive factor (RaRF), with a size of 81 kDa,
Antithrombin III (52 kDa),
Complement factor H precursor (143 kDa),
Albumin 1 (Fragment) (67 kDa) and
Mannose-binding lectin associated serine protease-2 (77 kDa).

Unexpectedly, all the binding partners correspond to proteins from the complement pathway, namely to human properdin, mannose-binding lectin C (MBL-C), MASP1, MASP2, antithrombin III, complement factor H and albumin. These surprising properties presently characterized of the polynucleotides or the corresponding polypeptides of International Applications WO2003/093316 and No. PCT/GB2004/004772 make them particularly suitable for the preparation of a medicament or of a pharmaceutical composition.

### Example 3

### Mouse Models

The biological activity of INSP052 polypeptides can be tested in mouse models of cardiovascular diseases as described in Chu et al. (Gene-engineered models for genetic manipulation and functional analysis of the cardiovascular system in mice. Chang Gung Med J. December 2003; 26(12):868-78.), in Ma et al. (Neurocardiovascular regulation in mice: experimental approaches and novel findings. Clin Exp Pharmacol Physiol. November 2003; 30(11):885-93.) or Svenson et al. (Invited review: Identifying new mouse models of cardiovascular disease: a review of high-throughput screens of mutagenized and inbred strains. J Appl Physiol. April 2003; 94(4):1650-9; discussion 1673.).

Alternatively, biological activity of INSP052 polypeptides can be tested in mouse models of infectious diseases as described in Cluff et al. (Synthetic toll-like receptor 4 agonists stimulate innate resistance to infectious challenge. Infect Immun. May 2005; 73(5):3044-52), Ahmed et al. (Mycetoma caused by Madurella mycetomatis: a neglected infectious burden. Lancet Infect Dis. September 2004; 4(9):566-74.), in Schriewer et al. (Mouse models for studying orthopoxvirus respiratory infections. Methods Mol Biol. 2004; 269:289-308.) or Davis and (Breaking the species barrier: use of SCID mouse-human chimeras for the study of human infectious diseases. Cell Microbiol. December 2003; 5(12):849-60.).

Alternatively, biological activity of INSP052 polypeptides can be tested in mouse models of glomerulonephritis as described in Kikuchi et al. (A transgenic mouse model of autoimmune glomerulonephritis and necrotizing arteritis associated with cryoglobulinemia. J Immunol. October 2002; 169(8):4644-50.), Nordstrand et al. (Streptokinase as a mediator of acute post-streptococcal glomerulonephritis in an experimental mouse model. Infect Immun. January 1998; 66(1):315-21.), or Rondeau E. ([A new model of extramembranous glomerulonephritis in the mouse after a single injection of anti-aminopeptidase A monoclonal antibodies] Nephrologie. 1992; 13(3):138.).

### REFERENCES

1. Abraham D J, Shiwen X, Black C M, Sa S, Xu Y, Leask A. J Biol Chem. 2000 May 19; 275(20):15220-5.

2. Altschul S F et al, J Mol Biol, 215, 403-410, 1990

3. Altschul S F et al, Nucleic Acids Res., 25:389-3402, 1997

4. Devereux J et al, Nucleic Acids Res, 12, 387-395, 1984.

5. Engelmann, H., Novick, D., and Wallach, D., 1990, J. Biol. Chem. 265, 1531-1536.

6. Pearson W R, Methods in Enzymology, 183, 63-99, 1990

7. Pearson W R and Lipman D J, Proc Nat Acad Sci USA, 85, 2444-2448, 1988.

8. Tucci, A., James, H., Chicheportiche, R., Bonnefoy, J. Y., Dayer, J. M., and Zubler, R. H., 1992, J. Immunol. 148, 2778-2784.

1-32. (canceled)

33. A method of treating a disease comprising administering to a patient in need thereof an effective amount of a composition comprising an INSP052 polypeptide and a pharmaceutically acceptable carrier, said INSP052 polypeptide:
a) consisting of SEQ ID NO: 16;
b) comprising SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 27, SEQ ID NO: 28, SEQ ID NO: 29, SEQ ID NO: 30, SEQ ID NO: 31, SEQ ID NO: 32 or SEQ ID NO: 33;
c) consisting of any of SEQ ID NO: 20, SEQ ID NO: 22, SEQ ID NO:27, SEQ ID NO: 28, SEQ ID NO: 29, SEQ ID NO: 30, SEQ ID NO: 31, SEQ ID NO: 32 or SEQ ID NO: 33 in a soluble form;
d) being a mature form of SEQ ID NO: 22, SEQ ID NO: 24 or SEQ ID NO: 26;
e) consisting of SEQ ID NO: 29;
f) being a glycosylated form of any of the polypeptides of (a) to (e), wherein the polypeptide is glycosylated at one or more sites;
g) comprising a mutein of any of the polypeptides of (a) to (f), wherein the amino acid sequence has at least 40% or 50% or 60% or 70% or 80% or 90% identity to at least one of the corresponding sequences in (a) to (f), and wherein said mutein retains INSP052 biological activity;
h) comprising a mutein of any of the polypeptides of (a) to (f) wherein any changes in the amino acid sequence are conservative amino acid substitutions to the amino acid sequences in (a) to (f), and wherein said mutein retains INSP052 biological activity; or
i) comprising a salt, isoform, fusion protein, functional derivative, active fraction or circularly permutated derivative of any of the polypeptides of (a) to (h); and
said disease being selected from infectious disease, properdin-related disease, MBL2-related disease, MASP1-related disease, MASP2-related disease, Antithrombin III-related disease, Complement factor H-related disease or Albumin-related disease.

34. The method according to claim 33, wherein the infectious disease is selected from a group consisting of Systemic Fungal Disease, Rickettsial Disease, Chlamydial Disease, Parasitic Infection, Viral Disease, Abscess, Human Immunodeficiency Virus Infection, Bacteremia, Septic Shock, Sexually Transmitted Disease, and Bacterial Disease.

35. The method according to claim 34, wherein the bacterial disease is selected from a disease caused by Gram-Positive Cocci, caused by Gram-Negative Aerobic Cocci, caused by Gram-Positive Bacilli, caused by Gram-Negative Bacilli, caused by Anaerobic Bacilli, caused by Spirochetes or caused by Mycobacteria.

36. The method according to claim 35, wherein the disease caused by Gram-Negative Aerobic Cocci is selected from the group consisting of meningitis, bacteremia, urethritis, cervicitis, proctitis, pharyngitis, salpingitis, epididymitis, gonorrheal infection, acute cacterial meningitis, and Meningococcal infection.

37. The method according to claim 34, wherein the Parasitic Infection is selected from the group consisting of Extraintestinal Protozoa infection, infection with Free-Living Amebas, Intestinal Protozoa infection, Nematode (Roundworm) Infection, Trematode (Fluke) infection, and Cestodes (Tapeworms) infection.

38. The method according to claim 34, wherein the Viral Disease is selected from the group consisting of Respiratory Viral Disease, Herpesvirus Infection, Central Nervous System Viral Disease, Arbovirus, and Arenavirus Disease.

39. The method according to claim 33, wherein the properdin-related disease is a glomerular disease.

40. The method according to claim 39, wherein the glomerular disease is selected from the group consisting of nephritic syndrome, nephrotic syndrome, primary glomerular disease, and secondary renal disease.

41. The method according to claim 40, wherein the primary glomerular disease is selected from the group consisting of minimal change disease, focal segmental glomerulosclerosis, membranous glomerulonelhritis, membranoproliferative glomerulonephritis, mesangial proliferative glomerulonephritis, IgA nephropathy, rapidly progressive glomerulonephritis, and fibrillary glomerulonephritis.

42. The method according to claim 40, wherein the nephritic syndrome is selected from the group consisting of hematuria, hypertension, renal insufficiency, edema, acute glomerulonephritis, transient glomerulonephritis, postinfectious glomerulonephritis, fulminant glomerulonephritis, rapidly progressive glomerulonephritis (RPGN), indolent glomerulonephritis, IgA nephropathy, crescentic glomerulonephritis, Pauci-immune RPGN, Immune complex RPGN, Anti-GBM antibody disease autoimmunity, primary renal hematuric-proteinuric syndrome, asymptomatic hematuric-proteinuric syndrome, chronic nephritic-proteinuric syndrome, chronic glomerulonephritis, and slowly progressive glomerular disease.

43. The method according to claim 33, wherein the INSP052 polypeptide is:
a) glycosylated at residues 2, 71, 105, 134, 139 and/or 156 of SEQ ID NO: 22;
b) fused to an immunoglobulin (Ig);
c) fused to a Fc region of an immunoglobulin;
d) SEQ ID NO: 30;
e) a functional derivative that includes at least one moiety attached to one or more functional groups; or
f) a functional derivative that includes a polyethylene moiety.

44. The method according to claim 33, wherein said composition further comprises an interferon, for simultaneous, sequential, or separate use.

45. The method according to claim 44, wherein the interferon is interferon-β.

46. The method according to claim 33, wherein pharmaceutical composition further comprises a Tumor Necrosis Factor (TNF) antagonist for simultaneous, sequential, or separate use.

47. The method according to claim 46, wherein the TNF antagonist is TBPI and/or TBPII.

48. The method according to claim 33, wherein the composition further comprises an anti-infectious agent and/or an anti-blood clotting agent for simultaneous, sequential, or separate use.

49. The method according to claim 48, wherein the anti-infectious agent is selected from the group consisting of pentamidine, antibiotics, colistine, aminoglycosides, and amphotericin B.

50. The method according to claim 47, wherein the anti-blood clotting agent is selected from the group consisting of nitrates, nitroglycerin, isosorbide dinitrate, isosorbide mononitrate, vitamin C or E, beta-blockers, propranolol, labetalol, acebutolol, atenolol, metoprolol, bisoprolol, carvedilol, anticoagulants, heparin, warfarin, anti-platelet drug, aspirin, glycoprotein IIb/IIIa receptor antagonists, clopidogrel, NSAIDs, enoxaparin, dalteparin, reviparin, abciximab, eptifibatide, lamifiban, tirofiban, abciximab, clopidogrel, ticlopidine, hirudin, bivalirudin, argatroban, danaparoid, statins, Angiotensin Converting Enzyme Inhibitors Angiotensin converting enzyme (ACE) inhibitors, ramipril, captopril, enalapril, lisinopril, fosinopril, calcium channel blockers, verapamil, nifedipine, nicardipine, amlodipine, diltiazem, bepridil, ranolazine, nicorandil, antibiotics, tetracyclines, quinolones, folic acid, thrombolytics, recombinant tissue plasminogen activators (rt-Pas), alteplase, activase, reteplase, fibrin-depleting agent, ancrod, batroxobin, thienopyridines, clopidogrel, ticlopidine, thienopyridines, direct thrombin inhibitors (DTIs), lepirudin, desirudin, inogatran, efegatran, ximelagatran, antifibrinolytics, tranexamic acid, and epsilon amino-caproic acid.

51. A method of treating a disease comprising administering to a patient in need thereof an effective amount of a composition comprising a nucleic acid encoding an INSP052 polypeptide and a pharmaceutically acceptable carrier, said INSP052 polypeptide:
a) consisting of SEQ ID NO: 16;
b) comprising SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 27, SEQ ID NO: 28, SEQ ID NO: 29, SEQ ID NO: 30, SEQ ID NO: 31, SEQ ID NO: 32 or SEQ ID NO: 33;
c) consisting of any of SEQ ID NO: 20, SEQ ID NO: 22, SEQ ID NO:27, SEQ ID NO: 28, SEQ ID NO: 29, SEQ ID NO: 30, SEQ ID NO: 31, SEQ ID NO: 32 or SEQ ID NO: 33 in a soluble form;
d) being a mature form of SEQ ID NO: 22, SEQ ID NO: 24 or SEQ ID NO: 26;
e) consisting of SEQ ID NO: 29;
f) being glycosylated form of any of the polypeptides of (a) to (e), wherein the polypeptide is glycosylated at one or more sites;
g) comprising a mutein of any of the polypeptides of (a) to (f), wherein the amino acid sequence has at least 40% or 50% or 60% or 70% or 80% or 90% identity to at least one of the corresponding sequences in (a) to (f), and wherein said mutein retains INSP052 biological activity;
h) comprising a mutein of any of the polypeptides of (a) to (f) wherein any changes in the amino acid sequence are conservative amino acid substitutions to the amino acid sequences in (a) to (f), and wherein said mutein retains INSP052 biological activity; or
i) comprising a salt, isoform, fusion protein, functional derivative, active fraction or circularly permutated derivative of any of the polypeptides of (a) to (h); and
said disease being selected from infectious disease, properdin-related disease, MBL2-related disease, MASP1-related disease, MASP2-related disease, Antithrombin III-related disease, Complement factor H-related disease or Albumin-related disease.

52. The method according to claim 51, wherein said nucleic acid encoding an INSP052 polypeptide is a vector.

53. The method according to claim 51, wherein said nucleic acid encoding an INSP052 polypeptide is integrated into a host cell.


SEQUENCE LISTING (33 sequences)

SEQ ID NO: 1 - DNA, 85 bp, homo sapiens
atgaagagagaaaggggagccctgtccagagcctccagggccctgcgccttgctccttttgtctaccttcttctgatccagacag

SEQ ID NO: 2 - protein, 29 aa, homo sapiens
MKRERGALSRASRALRLAPFVYLLLIQTD

SEQ ID NO: 3 - DNA, 342 bp, homo sapiens
accccctggagggggtgaacatcaccagccccgtgcgcctgatccatggcaccgtggggaagtcggctctgctttctgtgcagtacagcagtaccagcagcgacaggcctgtagtgaagtggcagctgaagcgggacaagccagtgaccgtggtgcagtccattggcacagaggtcatcggcaccctgcggcctgactatcgagaccgtatccgactctttgaaaatggctccctgcttctcagcgacctgcagctggccgatgagggcacctatgaggtcgagatctccatcaccgacgacaccttcactggggagaagaccatcaaccttactgtagatg

SEQ ID NO: 4 - protein, 114 aa, homo sapiens
PLEGVNITSPVRLIHGTVGKSALLSVQYSSTSSDRPVVKWQLKRDKPVTVVQSIGTEVIGTLRPDYRDRIRLFENGSLLLSDLQLADEGTYEVEISITDDTFTGEKTINLTVDV

SEQ ID NO: 5 - DNA, 282 bp, homo sapiens
tgcccatttcgaggccacaggtgttggtggcttcaaccactgtgctggagctcagcgaggccttcaccttgaactgctcacatgagaatggcaccaagcccagctacacctggctgaaggatggcaagcccctcctcaatgactcgagaatgctcctgtcccccgaccaaaaggtgctcaccatcacccgcgtgctcatggaggatgacgacctgtacagctgcatggtggagaaccccatcagccagggccgcagcctgcctgtcaagatcaccgtataca

SEQ ID NO: 6 - protein, 94 aa, homo sapiens
PISRPQVLVASTTVLELSEAFTLNCSHENGTKPSYTWLKDGKPLLNDSRMLLSPDQKVLTITRVLMEDDDLYSCMVENPISQGRSLPVKITVYR

SEQ ID NO: 7 - DNA, 94 bp, homo sapiens
gaagaagctccctttacatcatcttgtctacaggaggcatcttcctccttgtgaccttggtgacagtctgtgcctgctggaaaccctccaaaag

SEQ ID NO: 8 - protein, 31 aa, homo sapiens
RSSLYIILSTGGIFLLVTLVTVCACWKPSKR

SEQ ID NO: 9 - DNA, 74 bp, homo sapiens
gaaacagaagaagctagaaaagcaaaactccctggaatacatggatcagaatgatgaccgcctgaaaccagaag

SEQ ID NO: 10 - protein, 25 aa, homo sapiens
KQKKLEKQNSLEYMDQNDDRLKPEA

SEQ ID NO: 11 - DNA, 71 bp, homo sapiens
cagacaccctccctcgaagtggtgagcaggaacggaagaaccccatggcactctatatcctgaaggacaag

SEQ ID NO: 12 - protein, 23 aa, homo sapiens
DTLPRSGEQERKNPMALYILKDK

SEQ ID NO: 13 - DNA, 303 bp, homo sapiens
gactccccggagaccgaggagaacccggccccggagcctcgaagcgcgacggagcccggcccgcccggctactccgtgtctcccgccgtgcccggccgctcgccggggctgcccatccgctctgcccgccgctacccgcgctccccagcgcgctccccagccaccggccggacacactcgtcgccgcccagggccccgagctcgcccggccgctcgcgcagcgcctcgcgcacactgcggactgcgggcgtgcacataatccgcgagcaagacgaggccggcccggtggagatcagcgcctga

SEQ ID NO: 14 - protein, 100 aa, homo sapiens
DSPETEENPAPEPRSATEPGPPGYSVSPAVPGRSPGLPIRSARRYPRSPARSPATGRTHSSPPRAPSSPGRSRSASRTLRTAGVHIIREQDEAGPVEISA

SEQ ID NO: 15 - DNA, 1251 bp, homo sapiens
atgaagagagaaaggggagccctgtccagagcctccagggccctgcgccttgctccttttgtctaccttcttctgatccagacagaccccctggagggggtgaacatcaccagccccgtgcgcctgatccatggcaccgtggggaagtcggctctgctttctgtgcagtacagcagtaccagcagcgacaggcctgtagtgaagtggcagctgaagcgggacaagccagtgaccgtggtgcagtccattggcacagaggtcatcggcaccctgcggcctgactatcgagaccgtatccgactctttgaaaatggctccctgcttctcagcgacctgcagctggccgatgagggcacctatgaggtcgagatctccatcaccgacgacaccttcactggggagaagaccatcaaccttactgtagatgtgcccatttcgaggccacaggtgttggtggcttcaaccactgtgctggagctcagcgaggccttcaccttgaactgctcacatgagaatggcaccaagcccagctacacctggctgaaggatggcaagcccctcctcaatgactcgagaatgctcctgtcccccgaccaaaaggtgctcaccatcacccgcgtgctcatggaggatgacgacctgtacagctgcatggtggagaaccccatcagccagggccgcagcctgcctgtcaagatcaccgtatacagaagaagctccctttacatcatcttgtctacaggaggcatcttcctccttgtgaccttggtgacagtctgtgcctgctggaaaccctccaaaaggaaacagaagaagctagaaaagcaaaactccctggaatacatggatcagaatgatgaccgcctgaaaccagaagcagacaccctccctcgaagtggtgagcaggaacggaagaaccccatggcactctatatcctgaaggacaaggactccccggagaccgaggagaacccggccccggagcctcgaagcgcgacggagcccggcccgcccggctactccgtgtctcccgccgtgcccggccgctcgccggggctgcccatccgctctgcccgccgctacccgcgctccccagcgcgctccccagccaccggccggacacactcgtcgccgcccagggccccgagctcgcccggccgctcgcgcagcgcctcgcgcacactgcggactgcgggcgtgcacataatccgcgagcaagacgaggccggcccggtggagatcagcgcctga

SEQ ID NO: 16 - protein, 416 aa, homo sapiens
MKRERGALSRASRALRLAPFVYLLLIQTDPLEGVNITSPVRLIHGTVGKSALLSVQYSSTSSDRPVVKWQLKRDKPVTVVQSIGTEVIGTLRPDYRDRIRLFENGSLLLSDLQLADEGTYEVEISITDDTFTGEKTINLTVDVPISRPQVLVASTTVLELSEAFTLNCSHENGTKPSYTWLKDGKPLLNDSRMLLSPDQKVLTITRVLMEDDDLYSCMVENPISQGRSLPVKITVYRRSSLYIILSTGGIFLLVTLVTVCACWKPSKRKQKKLEKQNSLEYMDQNDDRLKPEADTLPRSGEQERKNPMALYILKDKDSPETEENPAPEPRSATEPGPPGYSVSPAVPGRSPGLPIRSARRYPRSPARSPATGRTHSSPPRAPSSPGRSRSASRTLRTAGVHIIREQDEAGPVEISA

SEQ ID NO: 17 - DNA, 1257 bp, mus musculus
atgaagagagaaaggggagccctgtcaagagcctccagggctctgcgcctctctccttttgtctacctgcttctcatccagccagtccccctggagggggtgaacatcaccagcccagtacgtctgatccacggcacagtggggaagtcggccctgctttccgtgcagtacagtagcaccagcagcgacaagcccgtggtgaagtggcagctgaagcgtgacaagccagtgaccgtggtgcagtctataggcacagaggtcattggcactctgcggcctgactatcgagaccgtatccggctctttgaaaatggctccttgcttctcagcgacctgcagctggcggatgagggaacctatgaagtggagatttccatcactgacgacaccttcaccggggagaagaccatcaacctcaccgtggatgtgcccatttcaaggccgcaggtattagtggcttcaaccactgtgctggagctcagtgaggccttcaccctcaactgctcccatgagaatggcaccaagcctagctacacgtggctgaaggatggcaaacccctcctcaatgactcccgaatgctcctgtcccctgaccaaaaggtgctcaccatcacccgagtactcatggaagatgacgacctgtacagctgtgtggtggagaaccccatcagccaggtccgcagcctgcctgtcaagatcactgtgtatagaagaagctccctctatatcatcttgtctacaggaggcatcttcctccttgtgaccctggtgacagtttgtgcctgctggaaaccctcaaaaaagtctaggaagaagaggaagttggagaagcaaaactccttggaatacatggatcagaatgatgaccgcctaaaatcagaagcagataccctaccccgaagtggagaacaggagcggaagaacccaatggcactctatatcctgaaggataaggattcctcagagccagatgaaaaccctgctacagagccacggagcaccacagaacccggtccccctggctactccgtgtcgccgcccgtgcccggccgctctccggggcttcccatccgctcagcccgccgctacccgcgctccccagcacgttcccctgccactggccggacgcacacgtcgccaccgcgggccccgagctcgccaggccgctcgcgcagctcttcgcgctcactgcggactgcaggcgtgcagagaatccgggagcaggacgagtcagggcaggtggagatcagtgcctga

SEQ ID NO: 18 - protein, 418 aa, mus musculus
MKRERGALSRASRALRLSPFVYLLLIQPVPLEGVNITSPVRLIHGTVGKSALLSVQYSSTSSDKPVVKWQLKRDKPVTVVQSIGTEVIGTLRPDYRDRIRLFENGSLLLSDLQLADEGTYEVEISITDDTFTGEKTINLTVDVPISRPQVLVASTTVLELSEAFTLNCSHENGTKPSYTWLKDGKPLLNDSRMLLSPDQKVLTITRVLMEDDDLYSCVVENPISQVRSLPVKITVYRRSSLYIILSTGGIFLLVTLVTVCACWKPSKKSRKKRKLEKQNSLEYMDQNDDRLKSEADTLPRSGEQERKNPMALYILKDKDSSEPDENPATEPRSTTEPGPPGYSVSPPVPGRSPGLPIRSARRYPRSPARSPATGRTHTSPPRAPSSPGRSRSSSRSLRTAGVQRIREQDESGQVEISA

SEQ ID NO: 19 - DNA, 720 bp, homo sapiens
atgaagagagaaaggggagccctgtccagagcctccagggccctgcgccttgctccttttgtctaccttcttctgatccagacagaccccctggagggggtgaacatcaccagccccgtgcgcctgatccatggcaccgtggggaagtcggctctgctttctgtgcagtacagcagtaccagcagcgacaggcctgtagtgaagtggcagctgaagcgggacaagccagtgaccgtggtgcagtccattggcacagaggtcatcggcaccctgcggcctgactatcgagaccgtatccgactctttgaaaatggctccctgcttctcagcgacctgcagctggccgatgagggcacctatgaggtcgagatctccatcaccgacgacaccttcactggggagaagaccatcaaccttactgtagatgtgcccatttcgaggccacaggtgttggtggcttcaaccactgtgctggagctcagcgaggccttcaccttgaactgctcacatgagaatggcaccaagcccagctacacctggctgaaggatggcaagcccctcctcaatgactcgagaatgctcctgtcccccgaccaaaaggtgctcaccatcacccgcgtgctcatggaggatgacgacctgtacagctgcatggtggagaaccccatcagccagggccgcagcctgcctgtcaagatcaccgtatacagaagaagctcc

SEQ ID NO: 20 - protein, 240 aa, homo sapiens
MKRERGALSRASRALRLAPFVYLLLIQTDPLEGVNITSPVRLIHGTVGKSALLSVQYSSTSSDRPVVKWQLKRDKPVTVVQSIGTEVIGTLRPDYRDRIRLFENGSLLLSDLQLADEGTYEVEISITDDTFTGEKTINLTVDVPISRPQVLVASTTVLELSEAFTLNCSHENGTKPSYTWLKDGKPLLNDSRMLLSPDQKVLTITRVLMEDDDLYSCMVENPISQGRSLPVKITVYRRSS

SEQ ID NO: 21 - DNA, 621 bp, homo sapiens
gtgaacatcaccagccccgtgcgcctgatccatggcaccgtggggaagtcggctctgctttctgtgcagtacagcagtaccagcagcgacaggcctgtagtgaagtggcagctgaagcgggacaagccagtgaccgtggtgcagtccattggcacagaggtcatcggcaccctgcggcctgactatcgagaccgtatccgactctttgaaaatggctccctgcttctcagcgacctgcagctggccgatgagggcacctatgaggtcgagatctccatcaccgacgacaccttcactggggagaagaccatcaaccttactgtagatgtgcccatttcgaggccacaggtgttggtggcttcaaccactgtgctggagctcagcgaggccttcaccttgaactgctcacatgagaatggcaccaagcccagctacacctggctgaaggatggcaagcccctcctcaatgactcgagaatgctcctgtcccccgaccaaaaggtgctcaccatcacccgcgtgctcatggaggatgacgacctgtacagctgcatggtggagaaccccatcagccagggccgcagcctgcctgtcaagatcaccgtatacagaagaagctcc

SEQ ID NO: 22 - protein, 207 aa, homo sapiens
VNITSPVRLIHGTVGKSALLSVQYSSTSSDRPVVKWQLKRDKPVTVVQSIGTEVIGTLRPDYRDRIRLFENGSLLLSDLQLADEGTYEVEISITDDTFTGEKTINLTVDVPISRPQVLVASTTVLELSEAFTLNCSHENGTKPSYTWLKDGKPLLNDSRMLLSPDQKVLTITRVLMEDDDLYSCMVENPISQGRSLPVKITVYRRSS

SEQ ID NO: 23 - DNA, 328 bp, homo sapiens
gtgaacatcaccagccccgtgcgcctgatccatggcaccgtggggaagtcggctctgctttctgtgcagtacagcagtaccagcagcgacaggcctgtagtgaagtggcagctgaagcgggacaagccagtgaccgtggtgcagtccattggcacagaggtcatcggcaccctgcggcctgactatcgagaccgtatccgactctttgaaaatggctccctgcttctcagcgacctgcagctggccgatgagggcacctatgaggtcgagatctccatcaccgacgacaccttcactggggagaagaccatcaaccttactgtagatg

SEQ ID NO: 24 - protein, 110 aa, homo sapiens
VNITSPVRLIHGTVGKSALLSVQYSSTSSDRPVVKWQLKRDKPVTVVQSIGTEVIGTLRPDYRDRIRLFENGSLLLSDLQLADEGTYEVEISITDDTFTGEKTINLTVDV

SEQ ID NO: 25 - DNA, 1152 bp, homo sapiens
gtgaacatcaccagccccgtgcgcctgatccatggcaccgtggggaagtcggctctgctttctgtgcagtacagcagtaccagcagcgacaggcctgtagtgaagtggcagctgaagcgggacaagccagtgaccgtggtgcagtccattggcacagaggtcatcggcaccctgcggcctgactatcgagaccgtatccgactctttgaaaatggctccctgcttctcagcgacctgcagctggccgatgagggcacctatgaggtcgagatctccatcaccgacgacaccttcactggggagaagaccatcaaccttactgtagatgtgcccatttcgaggccacaggtgttggtggcttcaaccactgtgctggagctcagcgaggccttcaccttgaactgctcacatgagaatggcaccaagcccagctacacctggctgaaggatggcaagcccctcctcaatgactcgagaatgctcctgtcccccgaccaaaaggtgctcaccatcacccgcgtgctcatggaggatgacgacctgtacagctgcatggtggagaaccccatcagccagggccgcagcctgcctgtcaagatcaccgtatacagaagaagctccctttacatcatcttgtctacaggaggcatcttcctccttgtgaccttggtgacagtctgtgcctgctggaaaccctccaaaaggaaacagaagaagctagaaaagcaaaactccctggaatacatggatcagaatgatgaccgcctgaaaccagaagcagacaccctccctcgaagtggtgagcaggaacggaagaaccccatggcactctatatcctgaaggacaaggactccccggagaccgaggagaacccggccccggagcctcgaagcgcgacggagcccggcccgcccggctactccgtgtctcccgccgtgcccggccgctcgccggggctgcccatccgctctgcccgccgctacccgcgctccccagcgcgctccccagccaccggccggacacactcgtcgccgcccagggccccgagctcgcccggccgctcgcgcagcgcctcgcgcacactgcggactgcgggcgtgcacataatccgcgagcaagacgaggccggcccggtggagatcagcgcctga

SEQ ID NO: 26 - protein, 383 aa, homo sapiens
VNITSPVRLIHGTVGKSALLSVQYSSTSSDRPVVKWQLKRDKPVTVVQSIGTEVIGTLRPDYRDRIRLFENGSLLLSDLQLADEGTYEVEISITDDTFTGEKTINLTVDVPISRPQVLVASTTVLELSEAFTLNCSHENGTKPSYTWLKDGKPLLNDSRMLLSPDQKVLTITRVLMEDDDLYSCMVENPISQGRSLPVKITVYRRSSLYIILSTGGIFLLVTLVTVCACWKPSKRKQKKLEKQNSLEYMDQNDDRLKPEADTLPRSGEQERKNPMALYILKDKDSPETEENPAPEPRSATEPGPPGYSVSPAVPGRSPGLPIRSARRYPRSPARSPATGRTHSSPPRAPSSPGRSRSASRTLRTAGVHIIREQDEAGPVEISA

SEQ ID NO: 27 - protein, 256 aa, homo sapiens
MKRERGALSRASRALRLAPFVYLLLIQTDPLEGVNITSPVRLIHGTVGKSALLSVQYSSTSSDRPVVKWQLKRDKPVTVVQSIGTEVIGTLRPDYRDRIRLFENGSLLLSDLQLADEGTYEVEISITDDTFTGEKTINLTVDVPISRPQVLVASTTVLELSEAFTLNCSHENGTKPSYTWLKDGKPLLNDSRMLLSPDQKVLTITRVLMEDDDLDSCVVENPINQGRTLPCKITVYKKSSLSSIWLQEAFSSLGPW

SEQ ID NO: 28 - protein, 256 aa, homo sapiens
MKRERGALSRASRALRLAPFVYLLLIQTDPLEGVNITSPVRLIHGTVGKSALLSVQYSSTSSDRPVVKWQLKRDKPVTVVQSIGTEVIGTLRPDYRDRIRLFENGSLLLSDLQLADEGTYEVEISITDDTFTGEKTINLTVDVPISRPQVLVASTTVLELSEAFTLNCSHENGTKPSYTWLKDGKPLLNDSRMLLSPDQKVLTITRVLMEDDDLDSCVVENPINQGRTLPCKITVYKKSSFYIICLKEASSSFGPW

SEQ ID NO: 29 - protein, 213 aa, homo sapiens
VNITSPVRLIHGTVGKSALLSVQYSSTSSDRPVVKWQLKRDKPVTVVQSIGTEVIGTLRPDYRDRIRLFENGSLLLSDLQLADEGTYEVEISITDDTFTGEKTINLTVDVPISRPQVLVASTTVLELSEAFTLNCSHENGTKPSYTWLKDGKPLLNDSRMLLSPDQKVLTITRVLMEDDDLYSCMVENPISQGRSLPVKITVYRRSSHHHHHH

SEQ ID NO: 30 - protein, 439 aa, homo sapiens [sequence omitted]

SEQ ID NO: 31 - protein, 186 aa, homo sapiens
VRLIHGTVGKSALLSVQYSSTSSDRPVVKWQLKRDKPVTVVQSIGTEVIGTLRPDYRDRIRLFENGSLLLSDLQLADEGTYEVEISITDDTFTGEKTINLTVDVPISRPQVLVASTTVLELSEAFTLNCSHENGTKPSYTWLKDGKPLLNDSRMLLSPDQKVLTITRVLMEDDDLYSCMVENPISQ

SEQ ID NO: 32 - protein, 131 aa, homo sapiens
VNITSPVRLIHGTVGSITDDTFTGEKTINLTVDVPISRPQVLVASTTVLELSEAFTLNCSHENGTKPSYTWLKDGKPLLNDSRMLLSPDQKVLTITRVLMEDDDLYSCMVENPISQGRSLPVKITVYRRSS

SEQ ID NO: 33 - protein, 150 aa, homo sapiens
VNITSPVRLIHGTVGKSALLSVQYSSTSSDRPVVKWQLKRDKPVTVVQSIGTEVIGTLRPDYRDRIRLFENGSLLLSDLQLADEGTYEVEISITDDTFTGEKTINLTVDVPISRPQVLVASTTVLELMVENPISQGRSLPVKITVYRRSS